(19) United States
(12) Patent Application Publication
(10) Pub. No.: US 2023/0220368 A1
(43) Pub. Date: Jul. 13, 2023

(54) COLD PROTEASE TREATMENT METHOD FOR PREPARING BIOLOGICAL SAMPLES

(71) Applicant: 10x Genomics, Inc., Pleasanton, CA (US)

(72) Inventor: Albert Kim, San Francisco, CA (US)

(21) Appl. No.: 17/938,562

(22) Filed: Oct. 6, 2022

Related U.S. Application Data
(63) Continuation of application No. PCT/US2021/026592, filed on Apr. 9, 2021.
Application No. PCT/US2021/026592 is a continuation of application No. 17/131,174, filed on Dec. 22, 2020.
(60) Provisional application No. 63/026,500, filed on May 18, 2020.
(60) Provisional application No. 63/008,591, filed on Apr. 10, 2020.

(51) Int. Cl. C12N 9/64; A01N 1/02

(52) U.S. Cl. CPC C12N 9/6408; C12Y 304/21; A01N 1/0205

(57) ABSTRACT

The present disclosure provides methods of preparing a fixed biological sample for use in an assay, wherein the method includes treatment of the sample with a low temperature active protease, optionally, in combination with an un-fixing reagent. The disclosure also provides assay methods, include partition-based methods, for fixed biological sample that use the low temperature protease treatment in combination with an un-fixing reagent. Kits comprising protease compositions, un-fixing agent compositions, and other assay reagents for use in the methods are also provided.

### CROSS-REFERENCE TO RELATED APPLICATIONS

This application is a continuation of International Application No. PCT/US2021/026592, filed on Sep. 4, 2021, which claims the benefit of priority to U.S. application Ser. No. 17/131,174, filed Dec. 22, 2020, U.S. Provisional Application No. 63/026,500, filed May 18, 2020, and to U.S. Provisional Application No. 63/008,591, filed Apr. 10, 2020, each of which is incorporated herein by reference in their entirety.

### FIELD

The present disclosure relates generally to methods for preparing a biological sample by incubating a fixed biological sample with a protease solution at low temperature, and optionally in combination with an un-fixing agent.

### BACKGROUND

Biological samples containing a variety of biomolecules can be processed for various purposes, such as detection of a disease (e.g., cancer) or genotyping (e.g., species identification). Microfluidic technologies have been developed for partitioning individual biological samples (e.g., cells) into discrete droplets. Each discrete droplet may be fluidically isolated from other droplets, enabling accurate control of respective environments in the droplets, allowing for each biological sample in a droplet to be processed separately. Biological samples in the discrete droplets can be barcoded and subjected to chemical or physical processes such as heating, cooling, or chemical reactions. This allows each discrete droplet to contain its own separate assay that can be qualitatively or quantitatively processed.

Biological samples are unstable. When a biological sample is removed from its viable niche physical decomposition begins immediately. The rate and degree of decomposition is determined by a number of factors including time, solution buffering conditions, temperature, source (e.g. certain tissues and cells a have higher levels of endogenous RNase activity), biological stress (e.g. enzymatic tissue dissociation can activate stress response genes), and physical manipulation (e.g. pipetting, centrifuging). The degradation includes important nucleic acid molecules (e.g., RNA), proteins, as well as higher-order 3D structure of molecular complexes, whole cells, tissues, organs, and organisms. The instability of biological samples is a significant obstacle for their use with partition-based assays (e.g., single cell assays). Sample degradation greatly limits the ability to use such assays accurately and reproducibly with a wide range of available biological samples.

The problem of biological sample instability can be mitigated by preserving or fixing the sample using standard biological preservation methods such as cryopreservation, dehydration (e.g., in methanol), high-salt storage (e.g., using RNAssist or RNAlater), and/or chemical fixing agents that create covalent crosslinks (e.g., paraformaldehyde or DSP). The ability to use such a fixed biological sample in an assay, particularly a single-cell assay, requires that the fixed biological sample can be rapidly and efficiently un-fixed so that the relevant assay can be carried out before sample degradation occurs.

A challenge in the preparation of biological samples, tissue or single cells, is how best to release a sufficient quantity of nucleic acids (e.g., RNA) from the fresh or fixed biological sample that are also of sufficient quality. Currently, heat is used in combination with lytic enzymes (such as proteases) and appears to be an important facilitator for the release of cells and nucleic acids. The heat, however, also can be a source of degradation due to RNA fragmentation and can also induce a heat-stress in fresh tissues that alters the natural RNA profile of the sample.

### SUMMARY

The present disclosure provides methods that allow the use of a low temperature protease treatment in the preparation biological samples for either bulk or single-cell assays, such as partition-based gene expression profiling assays.

In at least one embodiment, the present disclosure provides a method for preparing a biological sample comprising incubating a solution of a fixed biological sample and a protease at a temperature of between 5° C. and 15° C. for at least an hour. In another embodiment, the incubating is for between 1 h and 3 h.

In at least one embodiment of the method, the fixed biological sample has been fixed with paraformaldehyde (“PFA”); optionally, fixed with PFA at a concentration of 1%-4%.

In at least one embodiment of the method, the solution further comprises an un-fixing agent; optionally, the un-fixing agent is capable of removing crosslinks formed in biomolecules by fixation with PFA. In at least one embodiment, the un-fixing agent is a composition comprising a compound selected from compound (1), compound (2), compound (3), compound (4), compound (5), compound (6), compound (7), compound (8), compound (9), compound (10), compound (11), compound (12), compound (13), compound (14), compound (15), or a combination thereof; optionally, wherein the un-fixing agent is a composition comprising a compound selected from compound (1), compound (8), or a combination thereof.

In at least one embodiment of the method, the protease is a cold-active protease; optionally, wherein the protease has an average activity of at least 1.0 Units/mg of protease at a temperature of between about 5° C. and about 15° C. In at least one embodiment, the protease has maximum activity at a temperature of between about 50° C. and about 60° C.

In at least one embodiment of the method, subsequent to incubating, the solution is shaken at a temperature of between about 65° C. and 75° C. for at least 15 minutes.

In at least one embodiment of the method, the protease concentration in the solution is between about 1 mg/mL and 100 mg/mL; optionally, the protease concentration in the solution is between about 5 mg/mL and 10 mg/mL.

In at least one embodiment of the method, the protease is a serine protease (E.C. 3.4.21); optionally, wherein the serine protease is selected from chymotrypsin-like, trypsin-like, thrombin-like, elastase-like, and subtilisin-like. In at least one embodiment, the protease is selected from: alcalase, alkaline proteinase, ArcticZymes Proteinase, bacillopeptidase A, bacillopeptidase B, bioprase, colistinase, esperase, genenase, kazusase, maxatase, proteinase K, protease S, savinase, Serratia peptidase (i.e., peptidase derived from Serratia sp.), subtilisin A, subtilisin B, subtilisin BL, subtilisin E, subtilisin J, subtilisin S, subtilisin S41, thermoase, trypsin, and a combination thereof. In at least one embodiment, the protease is a non-naturally occurring protease.

In at least one embodiment of the method, the fixed biological sample is derived from a tissue sample, a biopsy sample, or a blood sample. In at least one embodiment, the fixed biological sample is a single cell.

In at least one embodiment of the method, the amount of time prior to incubating the solution when the biological sample is fixed is at least 1 hour, at least 2 hours, at least 6 hours, at least 12 hours, at least 24 hours, at least 1 week, at least 1 month, at least 6 months, or longer.

In at least one embodiment, the method further comprises generating a discrete droplet encapsulating the biological sample. In at least one embodiment, the method further comprises generating a discrete droplet encapsulating the fixed biological sample and the protease. In at least one embodiment, the method further comprises generating a discrete droplet encapsulating the fixed biological sample, the protease, and the un-fixing agent.

In at least one embodiment wherein the method comprises generating a discrete droplet, the discrete droplet further comprises assay reagents; optionally, wherein the assay reagents are contained in a bead. In at least one embodiment, the discrete droplet further comprises a barcode; optionally, wherein the barcode is contained in a bead.

In some embodiments, the methods for preparing a biological sample using a low temperature protease treatment as described above and elsewhere herein can be used in an assay method. In at least one embodiment present disclosure provides an assay method comprising: (a) preparing a biological sample by incubating a solution of a fixed biological sample, an un-fixing agent, and a protease at a temperature of between about 5° C. and about 15° C. for at least an hour; (b) contacting the biological sample with assay reagents; and (c) detecting analytes from the reaction of the assay reagents and the biological sample. In at least one embodiment, the assay method further comprises generating a discrete droplet encapsulating the biological sample and assay reagents. In another embodiment, the incubating is for between 1 h and 3 h.

In some embodiments, the methods for preparing a biological sample using a low temperature protease treatment as described above and elsewhere herein can be used in an assay method wherein the biological sample is recovered in a pellet. In at least one embodiment, the present disclosure further provides an assay method comprising: (a) incubating a solution comprising a fixed biological sample, an un-fixing agent, and a protease at a temperature of between about 5° C. and about 15° C. for at least an hour; (b) heating the solution of step (a) to 70 C for 15 minutes; (c) centrifuging the solution of step (b) to obtain a pellet comprising cells of an un-fixed biological sample; (d) resuspending the cells from the pellet in a solution; (e) generating a discrete droplet encapsulating a cell from the pellet of step (d) and assay reagents; and (e) detecting analytes from the reaction of the cell from the pellet and the assay reagent. In another embodiment, the incubating is for between 1 h and 3 h.

In some embodiments the present disclosure also provides a kit comprising materials useful in preparing a biological sample using a low temperature protease treatment and carrying out an assay method as described above and elsewhere herein. In at least one embodiment, the disclosure provides a kit comprising: assay reagents; an un-fixing agent composition; and a protease composition.

In at least one embodiment of the kit, the protease comprises a cold-active protease; optionally, wherein the protease of the composition has an average activity of at least 1.0 Units/mg of protease at a temperature of between about 5° C. and about 15° C. In at least one embodiment of the kit, the protease is selected from: alcalase, alkaline proteinase, ArcticZymes Proteinase, bacillopeptidase A, bacillopeptidase B, bioprase, colistinase, esperase, genenase, kazusase, maxatase, proteinase K, protease S, savinase, Serratia peptidase (i.e., peptidase derived from Serratia sp.), subtilisin A, subtilisin B, subtilisin BL, subtilisin E, subtilisin J, subtilisin S, subtilisin S41, thermoase, trypsin, and a combination thereof.

In at least one embodiment of the kit, the unfixing agent composition comprises a compound selected from compound (1), compound (2), compound (3), compound (4), compound (5), compound (6), compound (7), compound (8), compound (9), compound (10), compound (11), compound (12), compound (13), compound (14), compound (15), or a combination thereof.

In at least one embodiment of the kit, the un-fixing agent composition is contained in a bead. In at least one embodiment, the assay reagents are contained in a bead. In at least one embodiment, the assay reagents comprise a barcode.

In at least one embodiment of the kit, the kit further comprises a fixing reagent; optionally, wherein the fixing reagent is a solution of 1%-4% PFA.

### BRIEF DESCRIPTION OF THE FIGURES

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows an example of a microfluidic channel structure for partitioning individual biological particles.

FIG. 2 shows an example of a microfluidic channel structure for delivering barcode carrying beads to droplets.

FIG. 3 shows an example of a microfluidic channel structure for co-partitioning biological particles and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIG. 7 shows an exemplary barcode carrying bead.

FIG. 8 shows another exemplary barcode carrying bead.

FIG. 9 shows an exemplary microwell array schematic.

FIG. 10 shows an exemplary microwell array workflow for processing nucleic acid molecules.

FIG. 11 schematically illustrates examples of labelling agents.

FIG. 12 depicts an example of a barcode carrying bead.

FIG. 13A, FIG. 13B, and FIG. 13C schematically depict an example workflow for processing nucleic acid molecules.

FIG. 14A, FIG. 14B, and FIG. 14C depicts cDNA electropherogram plots from single-cell 3′-RT reactions. FIG. 14A: fresh PBMCs; FIG. 14B: 4% PFA fixed PBMCs treated only with ArcticZymes Proteinase; FIG. 14C: 4% PFA fixed PBMCs treated with ArcticZymes Proteinase and the un-fixing agent of compound (8), as described in Example 4.

FIG. 15 depicts plots of cell counting of different PBMC cell types found in fresh cells as compared to PFA-fixed cells subjected to the un-fixing treatment with ArcticZymes Proteinase and the un-fixing agent of compound (8), as described in Example 4.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of”.

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of these limits, ranges excluding (i) either or (ii) both of those included limits are also included in the disclosure. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library(hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present disclosure pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### A. Overview of Embodiments

Recognized herein is the need for methods, compositions, kits, and systems for analyzing multiple cellular analytes (e.g., genomic, epigenomic, transcriptomic, metabolomic, and/or proteomic information) from fixed biological samples, e.g., individual cells, a population of cells, tissue samples, and other kinds of biological samples. The ability to carry out an accurate assay of a biological sample requires the rapid and efficient release of the cellular analytes (e.g., mRNA transcripts) from the sample so that the relevant cellular analyte information can be obtained before degradation occurs. Ideally, the state of the cellular analytes released from the biological sample is not significantly altered relative to its natural environment, that is the state it is in the cell before the treatment to release it. Typical methods for releasing cellular analytes from a biological sample for use in an assay involves the use of some combination of lysis agents, enzymatic inhibitors, chelating agents, physical agitation, and heat to facilitate the activity of the various reagents involved.

The present disclosure provides methods that result in improved release of cellular analytes from fixed biological samples. The biological samples prepared using these methods allow improved assays, including partition-based assays, to be carried out using fixed samples and fewer or no artifacts in the cellular analyte information that is obtained. The methods involve the use of a protease treatment at low temperature, optionally, together with un-fixing agents, to release the cellular analytes from the fixed sample. The use of a low temperature protease treatment to release cellular analytes from a fixed biological sample (e.g., tissue or cell) is contrary to standard methods known in the art, which generally use proteases at 37° C. or even higher temperature. It is a surprising advantage of the methods of the present disclosure that incubating a fixed biological sample in solution with a protease at a temperature of between 5° C. and 15° C. for at least an hour provides a sample containing cellular analytes (e.g., mRNA) that yields improved results in assays for the characterization of the cellular analytes (e.g., RNAseq assay). The methods using low temperature protease treatment demonstrate additional advantages when used in combination with an un-fixing agent, such as an un-fixing agent capable of removing crosslinks formed by fixation with paraformaldehyde (e.g., the un-fixing agents of compounds (1)-(15)).

### B. Fixed Biological Samples

The ability to use a fixed biological sample in an assay requires rapid and efficient un-fixing of the sample so that the assay can be carried out and the relevant cellular analyte information obtained before degradation occurs. Ideally, the assay data obtained from an un-fixed biological sample should be identical to that obtained from a fresh sample, or resemble a sample obtained from its natural environment as closely as possible. The methods for biological sample preparation of the present disclosure using a low temperature protease treatment allows for the use of a previously fixed biological sample in an assay, such as a partition-based RNAseq assay.

The term “biological sample,” as used herein refers to any sample of biological origin that includes a biomolecule, such as a nucleic acid, a protein, a carbohydrate, and/or a lipid. Biological samples used in the methods of the disclosure include blood and other liquid samples of biological origin, solid tissue samples such as a tissue sample (i.e., tissue specimen), a biopsy (i.e., a biopsy specimen), or tissue cultures or cells derived therefrom and the progeny thereof. This includes samples that have been manipulated in any way after isolation from the biological source, such as by treatment with reagents (e.g., fixation reagents, thereby generating a fixed biological sample); samples such as tissues that are embedded in medium (e.g., paraffin); sectioned tissue sample (e.g., sectioned samples that are mounted on a solid substrate such as a glass slide); washed; or enrichment for certain cell populations, such as cancer cells, neurons, stem cells, etc. The term also encompasses samples that have been enriched for particular types of molecules, e.g., nucleic acids, polypeptides, etc. “Biological sample” encompasses a clinical sample, and also includes tissue obtained by surgical resection, tissue obtained by biopsy, cells in culture, cell supernatants, cell lysates, tissue samples (i.e., tissue specimens), organs, bone marrow, blood, plasma, serum, and the like. A “biological sample” also includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample having cells (e.g., cancer cells) from a patient.

It is contemplated that the biological samples used in the methods of the present disclosure can be derived from another sample. Biological samples can include a tissue sample, such as a biopsy, core biopsy, needle aspirate, or fine needle aspirate. Biological samples also include a biological fluid sample, such as a blood sample, urine sample, or saliva sample, or the biological sample may be a skin sample, a cheek swab. The biological sample may be a plasma or serum sample. The biological sample may include cells or be a cell-free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

A range of methods exist for preserving biological sample integrity, and limiting decomposition include cryopreservation, dehydration (e.g., methanol), high salt storage (e.g., using RNAssist, or RNAlater), and treatment with chemical fixing agents that typically create covalently crosslinks in the biomolecules of the sample (e.g., paraformaldehyde). These techniques for stabilizing biological samples can be used alone or in combination, and each can be reversed to various extents using un-fixing treatments.

The ability to prepare a biological sample for use in an assay starting from a fixed biological sample is a feature of the methods of the present disclosure. The term “fixed” as used herein with regard to biological samples refers the state of being preserved from decay and/or degradation. “Fixation” refers to a process that results in a fixed sample and can include contacting the biomolecules within a biological sample with a fixative (or fixation reagent) for some amount of time, whereby the fixative results in covalent bonding interactions such as crosslinks between biomolecules in the sample. A “fixed biological sample” refers to a biological sample that has been contacted with a fixation reagent. For example, a formaldehyde-fixed biological sample has been contacted with the fixation reagent formaldehyde. “Fixed cells” or “fixed tissues” refer to cells or tissues that have been in contact with a fixative under conditions sufficient to allow or result in the formation of intra- and inter-molecular covalent crosslinks between biomolecules in the biological sample.

Herein, “un-fixed” refers to the processed condition of a cell, a plurality of cells, a tissue sample or any other biological sample that is characterized by a prior state of fixation followed by a reversal of the prior state of fixation. For instance, an un-fixed cell may also be referred to as a “previously fixed” cell. In one embodiment, an un-fixed cell is characterized by broken or reversed covalent bonds in the biomolecules of the cell(s) or sample, where such covalent bonds were previously formed by treatment with a fixation agent (e.g., paraformaldehyde or PFA).

In one aspect, the present invention provides a method for analysis of fixed single cells. In one embodiment, the method comprises providing a plurality of fixed cells, wherein a fixed cell of said plurality of fixed cells comprises a plurality of crosslinked nucleic acid molecules. In another embodiment, the method further comprises un-fixing said fixed cell with a protease at a temperature of between about 5° C. and about 15° C. for at least an hour to provide an un-fixed cell comprising a plurality of un-crosslinked nucleic acid molecules from said plurality of crosslinked nucleic acid molecules. In another embodiment, the solution further comprises one or more un-fixing agents as described herein (e.g., compound (1) and/or compound (8)). In other embodiments, said plurality of crosslinked nucleic acid molecules comprises cross-linked ribonucleic acid (RNA) molecules and/or said plurality of un-crosslinked (or de-crosslinked) nucleic acid molecules comprises un-crosslinked (or de-crosslinked) RNA molecules. In one embodiment, the fixation step and/or the un-fixing step are performed in bulk, i.e., outside of partitions. In another embodiment, the plurality of fixed cells or un-fixed cells comprises labeled fixed cells or labeled un-fixed cells (as further described herein).

In one additional embodiment, the method further comprises generating a plurality of barcoded nucleic acid molecules from said plurality of un-crosslinked (or de-crosslinked) nucleic acid molecules and a plurality of nucleic acid barcode molecules. In another embodiment, the generating is performed in a plurality of partitions. In one other embodiment, the plurality of partitions is a plurality of droplets or a plurality of wells. In another embodiment, a barcoded nucleic acid molecule of said plurality of barcoded nucleic acid molecules comprises i) a sequence corresponding to an un-crosslinked nucleic acid molecule of said plurality of said un-crosslinked (or de-crosslinked) nucleic acid molecules or reverse complement thereof, and ii) a barcode sequence or reverse complement thereof. In one embodiment, said sequence corresponding to an un-crosslinked (or de-crosslinked) nucleic acid molecule is a sequence corresponding to an un-crosslinked (or de-crosslinked) RNA molecule. In other embodiments, the barcode sequence is a partition-specific barcode sequence. In another embodiment, a partition of said plurality of partitions comprises said un-fixed cell and a support comprising said plurality of nucleic acid barcode molecules. In other embodiments, the support is a bead (e.g., a gel bead).

The amount of time a biological sample is contacted with a fixative to provide a fixed biological sample depend on the temperature, the nature of the sample, and the fixative used. For example, a biological sample can be contacted by a fixation reagent for 72 or less hours (e.g., 48 or less hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes).

Generally, contact of biological sample (e.g., a cell) with a fixation reagent (e g., paraformaldehyde or PFA) results the formation of intra- and inter-molecular covalent crosslinks between biomolecules in the biological sample. In some cases, the fixation reagent, formaldehyde, is known to result in covalent aminal crosslinks within RNA, DNA, and/or protein molecules. Examples of fixation reagents include but are not limited to aldehyde fixatives (e.g, formaldehyde, also commonly referred to as “paraformaldehyde,” “PFA,” and “formalin”; glutaraldehyde; etc.), imidoesters, NHS (N-Hydroxysuccinimide) esters, and the like.

The formation of crosslinks in biomolecules (e g., proteins, RNA, DNA) due to fixation greatly reduces the ability to detect (e.g., bind to, amplify, sequence, hybridize to) the biomolecules in standard assay methods. Common techniques to remove the crosslinks induced by fixative reagents (e.g., heat, acid) can cause further damage to the biomolecules (e g., loss of bases, chain hydrolysis, cleavage, denaturation, etc.). Further description of the consequences of fixation of tissue samples and the benefits of removing adducts and/or crosslinks are described in U.S. Pat. No. 8,288,122, which is hereby incorporated by reference in its entirety. For example, the widely used fixative reagent, paraformaldehyde or PFA, fixes tissue samples by catalyzing crosslink formation between basic amino acids in proteins, such as lysine and glutamine. Both intra-molecular and inter-molecular crosslinks can form in the protein. These crosslinks can preserve protein secondary structure and also eliminate enzymatic activity in the preserved tissue sample.

The present invention provides methods, composition, kits, and systems for treating fixed biological sample in order to process cellular analytes. Suitable cellular analytes include, without limitation, intracellular and extracellular analytes. The cellular analyte may be a protein, a metabolite, a metabolic byproduct, an antibody or antibody fragment, an enzyme, an antigen, a carbohydrate, a lipid, a macromolecule, or a combination thereof (e.g., proteoglycan) or other biomolecule. The cellular analyte may be a nucleic acid molecule. The cellular analyte may be a deoxyribonucleic acid (DNA) molecule or a ribonucleic acid (RNA) molecule. The DNA molecule may be a genomic DNA molecule. The cellular analyte may comprise coding or non-coding RNA. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA.

In some instances, the cellular analyte is associated with an intermediary entity, wherein the intermediary entity is analyzed to provide information about the cellular analyte and/or the intermediary entity itself. For instance, an intermediary entity (e.g., an antibody) may be bound to an extracellular analyte (e.g., a cell surface receptor), where the intermediary entity is processed to provide information about the intermediary entity, the extracellular analyte, or both. In one embodiment, the intermediary entity comprises an identifier (e.g., a barcode molecule) that can be used to generate barcode molecules (e.g., droplet-based barcoding) as further described herein.

In some embodiments, the fixed biological samples used in the methods has been fixed by treatment with formaldehyde. The term “formaldehyde” when used in the context of a fixative also refers “paraformaldehyde” (or “PFA”) and “formalin”, both of which are terms with specific meanings related to the formaldehyde composition (e.g., formalin is a mixture of formaldehyde and methanol). Thus, a formaldehyde-fixed biological sample may also be referred to as formalin-fixed or PFA-fixed. Protocols and methods for the use of formaldehyde as a fixation reagent to prepare fixed biological samples are well known in the art, and can be used in the methods of the present disclosure. For example, suitable ranges of formaldehyde concentrations for use in preparing a fixed biological sample is 0.1 to 10%, 1-8%, 1-4%, 1-2%, 3-5%, or 3.5-4.5%. In at least one embodiment of the method for preparing a biological sample comprising incubating a solution of a fixed biological sample and a protease at a temperature of between 5° C. and 15° C., the biological sample is fixed using a final concentration of 1% formaldehyde, 4% formaldehyde, or 10% formaldehyde. Typically, the formaldehyde is diluted from a more concentrated stock solution—e.g., a 35%, 25%, 15%, 10%, 5% PFA stock solution.

In at least one embodiment of the method for preparing a biological sample comprising incubating a solution of a fixed biological sample and a protease at a temperature of between 5° C. and 15° C., the methods disclosed herein allow for the use of fixed biological samples derived from a tissue sample, a biopsy sample, or a blood sample, that have been fixed with paraformaldehyde, and can comprise a fixed biological sample of a single cell. The stabilizing effect of the fixatives and the efficient of the un-fixing agents disclosed herein allow for the amount of time of sample fixation prior to generating the discrete droplet to be at least 1 hour, at least 2 hours, at least 6 hours, at least 12 hours, at least 24 hours, at least 1 week, at least 1 month, at least 6 months, or longer.

### C. Un-Fixing Agents

Conditions for reversing the effects of fixing a biological sample are known in the art, however, these conditions tend to be harsh. See e.g., WO2001/46402; US2005/0014203A1, and US2009/0202998A1. For example, treatment of PFA-treated tissue samples includes heating to 60-70C in Tris buffer for several hours, and yet typically results in removal of only a fraction of the fixative-induced crosslinks. Furthermore, the harsh un-fixing treatment conditions can result in permanent damage to biomolecules, particularly nucleic acids, in the sample. Recently, less harsh un-fixing techniques and conditions have been proposed that utilize compounds capable of chemically reversing the crosslinks resulting from fixation. See e.g., Karmakar et al., “Organocatalytic removal of formaldehyde adducts from RNA and DNA bases,” Nature Chemistry, 7: 752-758 (2015); US 2017/0283860A1; and US 2019/0135774A1.

The terms “un-fixing agent” (or “de-crosslinking agent”) as used herein refer to a compound or composition that reverses fixation and/or removes the crosslinks within or between biomolecules in a sample caused by previous use of a fixation reagent. In some embodiments, un-fixing agents are compounds that act catalytically in removing crosslinks in a fixed sample. Exemplary compounds (1)-(15) useful as un-fixing agents in the methods of the present disclosure include the compounds of Table 1 below.

TABLE 1
Exemplary Un-fixing Agent Compounds

| | |
|---|---|
| 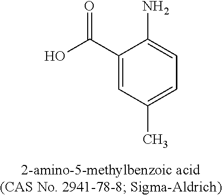 | (1) |
| 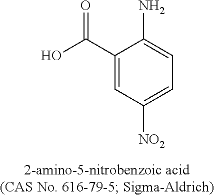 [SMILES: Nc1ccc([N+](=O)[O-])cc1C(=O)O] | (2) |
| 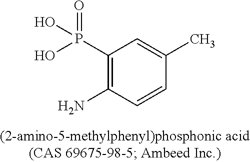 | (3) |
| 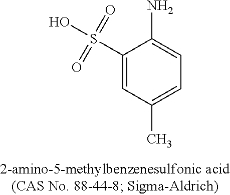 | (4 |
| 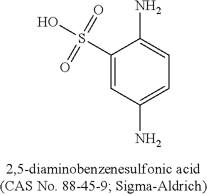 [SMILES: Nc1ccc(N)c(S(=O)(=O)O)c1] | (5) |
| 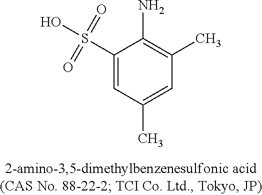 | (6) |
| 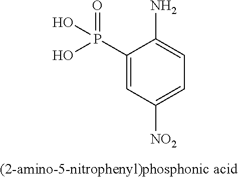 [SMILES: Nc1ccc([N+](=O)[O-])cc1P(=O)(O)O] | (7) |
| 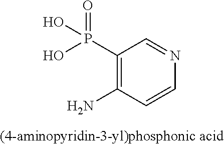 [SMILES: Nc1ccncc1P(=O)(O)O] | (8) |
| 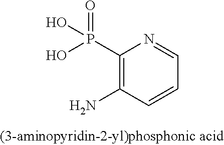 [SMILES: Nc1cccnc1P(=O)(O)O] | (9) |
| 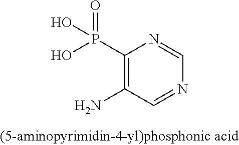 [SMILES: Nc1cncnc1P(=O)(O)O] | (10) |
| 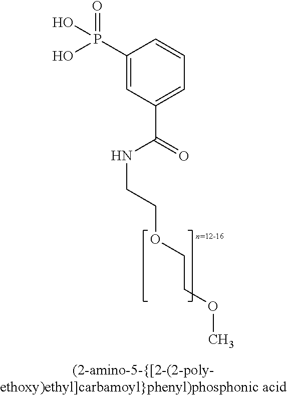 | (11) |
| 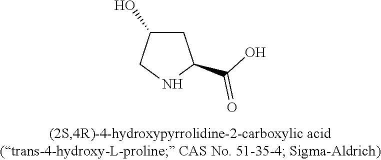 | (12) |
| 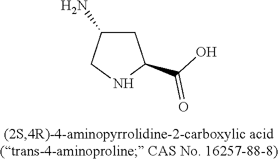 | (13) |
| 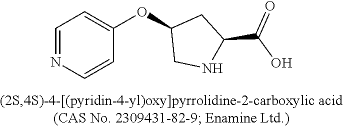 [SMILES: O=C(O)[C@@H]1C[C@H](Oc2ccncc2)CN1] | (14) |
| 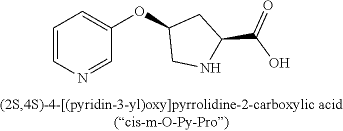 [SMILES: O=C(O)[C@@H]1C[C@H](Oc2cccnc2)CN1] | (15) |

At least one of the un-fixing agents of Table 1, compound (3), has previously been shown to catalytically break down the aminal and hemi-aminal adducts that form in RNA treated with formaldehyde, and are compatible with many RNA extraction and detection conditions. See e.g., Karmakar et al., “Organocatalytic removal of formaldehyde adducts from RNA and DNA bases,” Nature Chemistry, 7: 752-758 (2015); and US 2017/0283860A1.

Proline is a unique amino acid that contains a secondary amine in a 5-membered ring, resulting in high nucleophilicity. The high nucleophilicity together with a proximal amine or acid moiety in the proline analog structures of compounds (12), (13), (14), and (15) suggests that these compounds, like the compounds (1)-(11), also can be used as catalytically break down the aminal and hemi-aminal adducts that form in formaldehyde-fixed RNA and other biomolecules.

Compounds (1)-(6), (12), and (14) are commercially available. The compounds (7), (8), (9), (10), (11), (13), and (15) can be prepared from commercially available reagents using standard chemical synthesis techniques well-known in the art. See e.g., Crisalli et al., “Importance of ortho Proton Donors in Catalysis of Hydrazone Formation,” Org. Lett. 2013, 15, 7, 1646-1649.

Compounds (8) and (11) can be prepare by 2-step and 4-step syntheses, respectively, as described in Example 1. Briefly, in preparing compound (8), the compound, diethyl (4-aminopyridin-3-yl)phosphonate is prepared according to the procedure described in Guilard, R. et al. Synthesis, 2008, 10, 1575-1579. Then, the target compound (8), (4-aminopyridin-3-yl)phosphonic acid) is prepared by acid hydrolysis of the precursor compound of the diethyl (4-aminopyridin-3-yl)phosphonate. Compounds (9) and (10) can be prepared from similarly straightforward procedures. For example, compound (9) can be prepared in 2-steps from 2-bromopyridin-3-amine (CAS Reg. #39856-58-1; Sigma-Aldrich, St. Louis, Mo.) as shown in the scheme below.

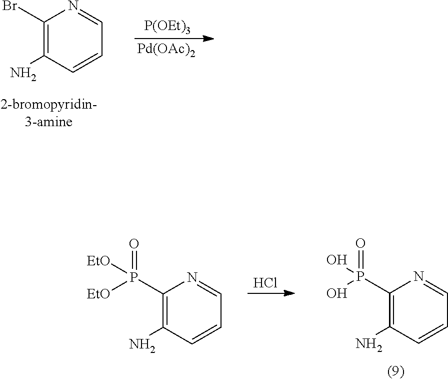

Compound (10) is prepared similarly in 2-steps from 4-bromopyrimidin-5-amine (CAS Reg. #849353-34-0; Ambeed, Inc., Arlington Heights, Ill., USA) as shown in the scheme below.

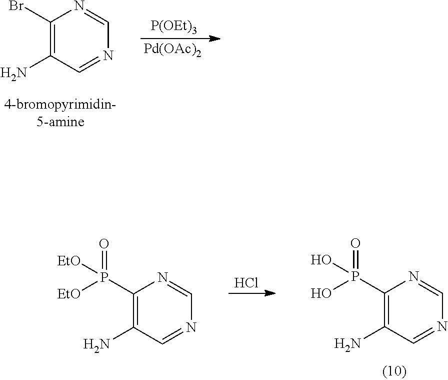

The proline analog compounds (13) and (15) are prepared via a straightforward single step deprotection from commercially available protected precursor compounds as described in Example 10.

Accordingly, in some embodiments of the methods of the present disclosure, the un-fixing agent used in the composition or method can comprise a compound selected from Table 1. For example, the un-fixing agent can comprise a compound of any of compound (1), compound (2), compound (3), compound (4), compound (5), compound (6), compound (7), compound (8), compound (9), compound (10), compound (11), compound (12), compound (13), compound (14), compound (15), or a combination of one or more the compounds of Table 1.

In at least one embodiment of the method for preparing a biological sample comprising incubating a solution of a fixed biological sample and a protease at a temperature of between 5° C. and 15° C., the incubation solution comprising the protease composition further comprises an un-fixing agent. In some embodiments, the biological sample is fixed with PFA and the un-fixing agent used in the solution is capable of removing crosslinks formed in biomolecules by fixation with PFA. In at least one embodiment, the un-fixing agent is a composition comprising a compound selected from compound (1), compound (2), compound (3), compound (4), compound (5), compound (6), compound (7), compound (8), compound (9), compound (10), compound (11), compound (12), compound (13), compound (14), compound (15), or a combination thereof; optionally, wherein the un-fixing agent is a composition comprising a compound selected from compound (1), compound (8), or a combination thereof.

### D. Low-Temperature Protease Treatment

The method for preparing a biological sample of the present disclosure comprises incubating a solution of a fixed biological sample with a protease at a temperature of between 5° C. and 15° C., can be carried using a wide range of protease that are active at low temperature. It is a surprising advantage of the method that it can be carried out using a protease that exhibits maximum activity in a much higher temperature range. For example, subtilisin A (Bacillus licheniformis), which exhibits maximum activity at between 55° C. and 60° C., can be used in the methods.

A wide range of proteases are known in the art for use as lysing agents and for releasing cellular analytes from cells, tissue samples, and other types of biological samples. As noted above, these proteases are used in methods carried out at room temperature or above, typically at a temperature of 37° C. or higher. In context of the methods of the present disclosure, it is contemplated that any protease that is cold-active (or psychrophilic) can be used. Cold-active proteases exhibits at least some measurable proteolytic activity at temperatures as low as 0 C, and typically exhibit significant proteolytic activity in the range of between about 5° C. and about 15° C. As noted above, even proteases that exhibit peak activity at much higher temperatures, such as subtilisin A, can have sufficient low temperature activity to be used as a cold-active protease in the methods of the present disclosure. In at least one embodiment of the present methods, the protease has an average activity at a temperature of between about 5° C. and about 15° C. of at least 1.0 U/mg, at least 5.0 U/mg, at 10.0 U/mg, at least 50 U/mg, at least 100 U/mg, or greater average activity. Determination of average protease activity in the temperature of between about 5° C. and about 15° C. can be carried out by the ordinary artisan using e.g., the well-known universal protease activity assay using casein substrate and Folin-Ciocalteu reagent. Reagents and kits for carrying out such protease activity assays are available commercially (e.g., from Millipore-Sigma; USA).

Accordingly, in at least one embodiment of the method, the protease used in the method is a cold-active protease; optionally, wherein the protease has an average activity of at least 1.0 Units/mg of protease at a temperature of between about 5° C. and about 15° C. In some embodiments, the protease has maximum activity at a temperature of between about 50° C. and about 60° C.

Additionally, in some embodiments of the method, it is contemplated that the temperature and time of incubation can be varied somewhat based on the particular protease used and that such conditions can be optimized by one of ordinary skill. Thus, in at least one embodiment, the method can be carried out at a temperature of between about 5° C. and about 13° C., between about 5° C. and about 10° C., between about 5° C. and about 8° C., between about 8° C. and about 15° C., or at a temperature of about 5° C., about 6° C., about 7° C., about 8° C., about 9° C., about 10° C., about 11° C., about 12° C., about 13° C., about 14° C., or about 15° C.

It is also contemplated that the amount of protease used in the low temperature treatment can be varied in order to adjust the low temperature proteolytic activity to an effective level. Accordingly, in at least one embodiment of the method, the protease concentration in the solution is between about 1 mg/mL and 100 mg/mL; optionally, the protease concentration in the solution is between about 5 mg/mL and 10 mg/mL.

In at least one embodiment of the method, the protease is a serine protease (E.C. 3.4.21); optionally, wherein the serine protease is selected from chymotrypsin-like, trypsin-like, thrombin-like, elastase-like, and subtilisin-like. A wide range of different serine proteases are well-characterized and commercially available. Among the serine proteases that may be useful in the methods of the present selected are: alcalase, alkaline proteinase, ArcticZymes Proteinase (ArcticZymes Technologies ASA, Tromso, Norway), bacillopeptidase A, bacillopeptidase B, bioprase, colistinase, esperase, genenase, kazusase, maxatase, proteinase K, protease S, savinase, Serratia peptidase (i.e., peptidase derived from Serratia sp.), subtilisin A, subtilisin B, subtilisin BL, subtilisin E, subtilisin J, subtilisin S, subtilisin S41, thermoase, and trypsin.

Proteases have differing substrate preferences and so mixtures of proteases are often used to release cellular analytes or other biological material from cells. Accordingly, in some embodiments it is contemplated that the low temperature protease treatment can comprise incubating the fixed biological sample with protease composition. In at least one embodiment, the method of the present disclosure can be carried out wherein the biological sample is incubated with a low-temperature active protease composition comprising at least two different proteases. In some embodiments, the composition comprises at least two proteases selected from: alcalase, alkaline proteinase, ArcticZymes Proteinase, bacillopeptidase A, bacillopeptidase B, bioprase, colistinase, esperase, genenase, kazusase, maxatase, proteinase K, protease S, savinase, Serratia peptidase (i.e., peptidase derived from Serratia sp.), subtilisin A, subtilisin B, subtilisin BL, subtilisin E, subtilisin J, subtilisin S, subtilisin S41, thermoase, and trypsin. For example, in at least one embodiment, a low-temperature active protease composition useful in the methods of the present disclosure comprises subtilisin A and proteinase K.

Although a wide-range of naturally-occurring proteases exhibit sufficient low temperature activity at a temperature of between about 5° C. and about 15° C. to be used in the methods of the present disclosure, it is also contemplated that non-naturally occurring (or engineered) low-temperature active proteases can be used in the methods of the disclosure. For example, a naturally occurring protease can be engineered using well-known methods of directed evolution to have a better activity profile over a desired temperature for certain types of biological sample preparation conditions. Accordingly, in at least one embodiment of the methods of the present disclosure, the protease is a non-naturally occurring protease.

The low-temperature protease treatment used in the methods for preparing a biological sample from a previously fixed biological sample generally comprises incubating the sample in an aqueous solution containing the protease at a temperature of between about 5° C. and about 15° C. for at least an hour. In another embodiment, the incubating is for between 1 h and 3 h. In some embodiments, the solution further comprises an un-fixing agent that reverses crosslinks between biomolecules of the sample during the low-temperature incubation period. It is also contemplated that in some embodiments, a short period of heating and physical agitation of the sample applied subsequent to the incubation can assist in the sample preparation process without creating artifacts associated with standard high-temperature protease treatments. Accordingly, in at least one embodiment, the method of the present disclosure can be carried out wherein subsequent to incubating the solution is shaken at a temperature of between about 65° C. and 75° C. for at least 15 minutes.

### E. Uses in Assay Methods

The methods of the present disclosure that use low temperature protease treatment of fixed biological samples can be used to prepare samples for use in assay methods. Such assay methods can include “bulk” assays with relatively large sample sizes, or single-cell assays, such as partition-based (or droplet-based) assays.

In some embodiments, the methods for preparing a biological sample using a low temperature protease treatment as described above and elsewhere herein can be used in an assay method. In at least one embodiment present disclosure provides an assay method comprising: (a) preparing a biological sample by incubating a solution of a fixed biological sample, an un-fixing agent, and a protease at a temperature of between about 5° C. and about 15° C. for at least an hour; (b) contacting the biological sample with assay reagents; and (c) detecting analytes from the reaction of the assay reagents and the biological sample. In at least one embodiment, the assay method further comprises generating a discrete droplet encapsulating the biological sample and assay reagents. In another embodiment, the incubating is for between 1 h and 3 h.

In some embodiments, the methods for preparing a biological sample using a low temperature protease treatment as described above and elsewhere herein can be used in an assay method wherein the biological sample is recovered in a pellet. In at least one embodiment, the present disclosure further provides an assay method comprising: (a) incubating a solution comprising a fixed biological sample, an un-fixing agent, and a protease at a temperature of between about 5° C. and about 15° C. for at least an hour; (b) heating the solution of step (a) to 70 C for 15 minutes; (c) centrifuging the solution of step (b) to obtain a pellet comprising cells of an un-fixed biological sample; (d) resuspending the cells from the pellet in a solution; (e) generating a discrete droplet encapsulating a cell from the pellet of step (d) and assay reagents; and (e) detecting analytes from the reaction of the cell from the pellet and the assay reagent. In another embodiment, the incubating is for between 1 h and 3 h.

### F. Use in Partition-Based Sample Preparation and Assay Methods

The use of fixed biological samples in partition-based assays creates additional challenges due to the small sample amounts and the need to carry out the assay with an extremely small sample volume while maintaining physical separation of the sample. The term “partition,” as used herein, generally, refers to a space or volume that may be suitable to contain one or more species or conduct one or more reactions. A partition may be a physical compartment, such as a droplet or well (e.g., a microwell). The partition may isolate space or volume from another space or volume. The partition may be a droplet of a first phase (e.g., aqueous phase) in a second phase (e.g., oil) that is immiscible with the first phase. The partition may be a droplet of a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

Preparation of a partition containing a biological sample that is useful in a partition-based assay involves numerous steps (e.g., sample transport, tissue dissociation, liquid phase washing and transfer, library preparation) that typically take from a few hours to days. During this preparation time an un-fixed biological sample will begin to degrade, and decompose resulting in significant loss of cellular analyte information and thus yield assay results that do not reflect the natural state of the sample.

One type of partition-based assay is a droplet-based assay. Such assays use a biological sample that is isolated and partitioned in discrete droplet in an emulsion. The discrete droplet typically includes a unique identifier for the sample in the form of a unique oligonucleotide sequence also contained in the droplet. The discrete droplet can also contain the assay reagents that are used to generate detectable analytes (e.g., 3′ cDNA sequences) from the sample and provide useful information about it (e.g., RNA transcript profile).

The methods of the present disclosure are useful to prepare a biological sample from a fixed biological sample encapsulated in discrete droplet along with low-temperature active protease, and an un-fixing agent. The combination of the protease and the un-fixing agent with the fixed sample in the droplet are capable of reversing the fixed state of the biomolecules in the sample while it is sequestered in the droplet. Accordingly, in some embodiments, the present disclosure provides a method for preparing a biological sample comprising: generating a discrete droplet encapsulating a fixed biological sample, a protease composition, and an un-fixing agent. This method can further comprise a step of fixing the biological sample prior to generating the discrete droplet.

In at least one embodiment, the method further comprises generating a discrete droplet encapsulating the biological sample. In at least one embodiment, the method further comprises generating a discrete droplet encapsulating the fixed biological sample and the protease. In at least one embodiment, the method further comprises generating a discrete droplet encapsulating the fixed biological sample, the protease, and the un-fixing agent.

In at least one embodiment wherein the method comprises generating a discrete droplet, the discrete droplet further comprises assay reagents; optionally, wherein the assay reagents are contained in a bead. In at least one embodiment, the discrete droplet further comprises a barcode; optionally, wherein the barcode contained in a bead.

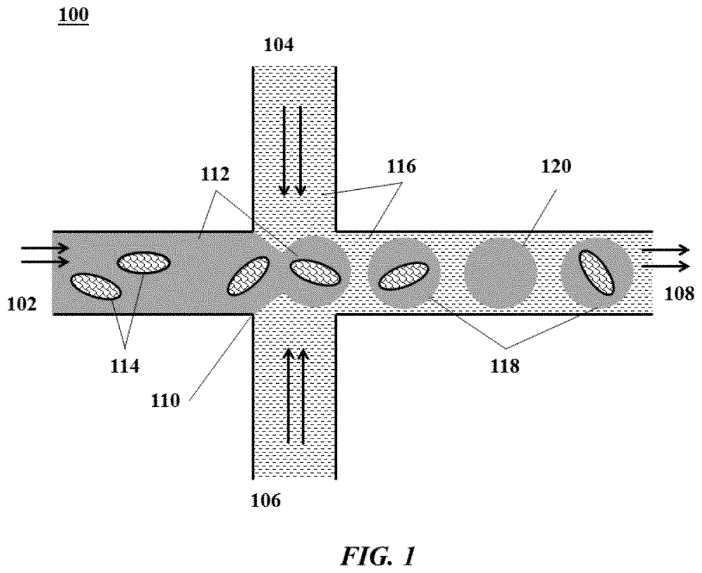

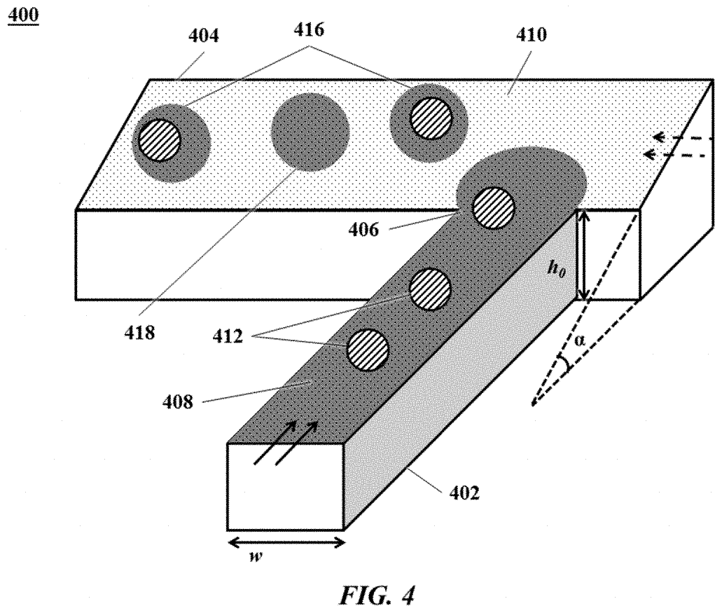

Methods, techniques, and protocols useful for partitioning biological samples (e.g., individual cells, biomolecular contents of cells, etc.) into discrete droplets are known and well described in the art. The discrete droplets generated act a nanoliter-scale container that can maintain separation the droplet contents from the contents of other droplets in the emulsion. Methods and systems for creating stable discrete droplets encapsulating individual particles from biological samples in non-aqueous or oil emulsions are described in, e.g., U.S. Patent Application Publication Nos. 2010/0105112 and 2019/0100632, each of which is entirely incorporated herein by reference for all purposes. Briefly, discrete droplets in an emulsion encapsulating a biological sample is accomplished by introducing a flowing stream of an aqueous fluid containing the biological sample into a flowing stream of a non-aqueous fluid with which it is immiscible, such that droplets are generated at the junction of the two streams (see FIGS. 1-3). By providing the aqueous stream at a certain concentration and/or flow rate of the biological sample, the occupancy of the resulting droplets can be controlled. For example, the relative flow rates of the immiscible fluids can be selected such that, on average, the discrete droplet each contains less than one biological particle. Such a flow rate ensures that the droplets that are occupied are primarily occupied by a single sample (e.g., a single cell). Discrete droplets in an emulsion encapsulating a biological sample is also accomplished using a microfluidic architecture comprising a channel segment having a channel junction with a reservoir (see FIGS. 4-6).

The term “biological particle,” as used herein, generally refers to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle.

In some cases, the droplets among a plurality of discrete droplets formed in the manner contain at most one particle (e.g., one bead, one cell). The flows and microfluidic channel architectures also can be controlled to ensure a given number of singly occupied droplets, less than a certain level of unoccupied droplets, and/or less than a certain level of multiply occupied droplets.

In another aspect of the disclosure, fixed cells, protease composition, and optional un-fixing agent composition may then be partitioned (e.g., in a droplet or well) with other reagents for processing of one or more analytes as described herein. In one embodiment, the fixed cell, protease composition, and optional un-fixing agent composition may be partitioned with a support (e.g., a bead) comprising nucleic acid molecules suitable for barcoding of the one or more analytes. In another embodiment, the nucleic acid molecules may include nucleic acid sequences that provide identifying information, e.g., barcode sequence(s).

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence (e.g., targeted by a primer sequence) or a non-targeted sequence. For example, in the methods, compositions, kits, and systems described herein, hybridization and reverse transcription of the nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the mRNA.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

FIG. 1 shows an exemplary microfluidic channel structure 100 useful for generating discrete droplets encapsulating a particle from a biological sample, such as a single cell. The channel structure 100 can include channel segments 102, 104, 106 and 108 communicating at a channel junction 110. In operation, a first aqueous fluid 112 that that includes suspended particles (e.g., cells) from a biological sample 114 are transported along channel segment 102 into junction 110, while a second fluid 116 (or “partitioning fluid”) that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual particle from a biological sample 114 (such as droplet 118), or discrete droplet can be generated that includes more than one particle 114 (not shown in FIG. 1). A discrete droplet may contain no biological particle 114 (such as droplet 120). Each discrete droplet is capable of maintaining separation of its own contents (e.g., individual biological particle 114) from the contents of other droplets.

Typically, the second fluid 116 comprises an oil, such as a fluorinated oil, that includes a fluoro-surfactant that helps to stabilize the resulting droplets. Examples of useful partitioning fluids and fluoro-surfactants are described in e.g., U.S. Patent Application Publication No. 2010/0105112, which is entirely incorporated herein by reference for all purposes.

The microfluidic channels for generating discrete droplets as exemplified in FIG. 1 may be coupled to any of a variety of different fluid sources or receiving components, including reservoirs, tubing, manifolds, or fluidic components of other systems. Additionally, the microfluidic channel structure 100 may have other geometries, including geometries having more than one channel junction. For example, the microfluidic channel structure can have 2, 3, 4, or 5 channel segments each carrying biological particles from a biological sample, assay reagents, and/or beads that meet at a channel junction.

Generally, the fluids used in generating the discrete droplets are directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electro-kinetic pumping, vacuum, capillary or gravity flow, or the like.

One of ordinary skill will recognize that numerous different microfluidic channel designs are available that can be used with the methods of the present disclosure to provide discrete droplets containing a biological particle from a fixed biological sample, a protease composition, an un-fixing agent composition, and/or a bead with a barcode and/or other assay reagents.

The inclusion of a barcode in a discrete droplet along with the biological sample provides a unique identifier that allows data from the biological sample to be distinguished and individually analyzed. Barcodes can be delivered previous to, subsequent to, or concurrent with the biological sample in discrete droplet. For example, barcodes may be injected into droplets previous to, subsequent to, or concurrently with droplet generation. Barcodes useful in the methods of the present disclosure typically comprise a nucleic acid molecule (e.g., an oligonucleotide). The nucleic acid barcode molecules typically are delivered to a partition via a support, such as a bead. In some cases, barcode nucleic acid molecules are initially associated with the bead upon generation of the discrete droplet, and then released from the bead upon application of a stimulus to droplet. Barcode carrying beads useful in the methods of the present disclosure are described in further detail elsewhere herein.

Methods and systems for partitioning barcode carrying beads into droplets are provided in US Patent Nos. 10480029, 10858702, and 10725027, US. Patent Publication Nos. 2019/0367997 and 2019/0064173, and International Application Nos. PCT/US20/17785 and PCT/US20/020486, each of which is herein entirely incorporated by reference for all purposes.

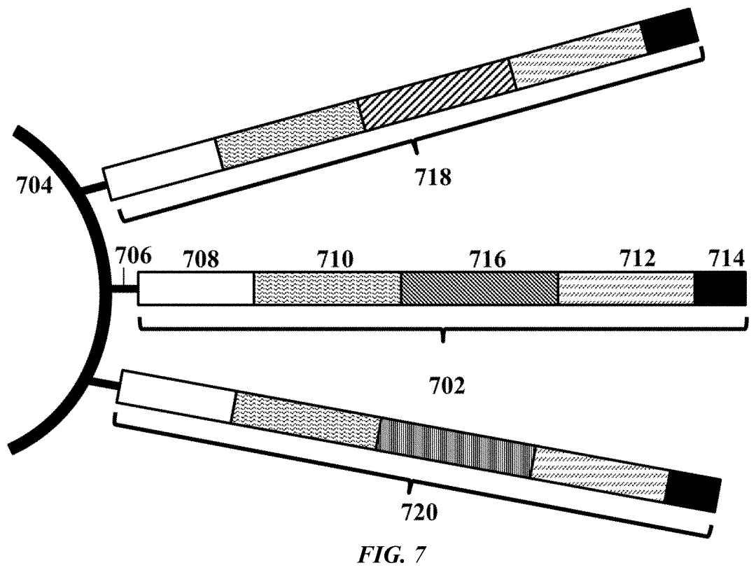

FIG. 7 illustrates an example of a barcode carrying bead. A nucleic acid molecule 702, such as an oligonucleotide, can be coupled to a bead 704 by a releasable linkage 706, such as, for example, a disulfide linker. The same bead 704 may be coupled (e.g., via releasable linkage) to one or more other nucleic acid molecules 718, 720. The nucleic acid molecule 702 may be or comprise a barcode. As noted elsewhere herein, the structure of the barcode may comprise a number of sequence elements. The nucleic acid molecule 702 may comprise a functional sequence 708 that may be used in subsequent processing. For example, the functional sequence 708 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 702 may comprise a barcode sequence 710 for use in barcoding the sample (e.g., DNA, RNA, protein, antibody, etc.). In some cases, the barcode sequence 710 can be bead-specific such that the barcode sequence 710 is common to all nucleic acid molecules (e.g., including nucleic acid molecule 702) coupled to the same bead 704. Alternatively or in addition, the barcode sequence 710 can be partition-specific such that the barcode sequence 710 is common to all nucleic acid molecules coupled to one or more beads that are partitioned into the same partition. The nucleic acid molecule 702 may comprise a specific priming sequence 712, such as an mRNA specific priming sequence (e.g., poly-T sequence), a targeted priming sequence, and/or a random priming sequence. The nucleic acid molecule 702 may comprise an anchoring sequence 714 to ensure that the specific priming sequence 712 hybridizes at the sequence end (e.g., of the mRNA). For example, the anchoring sequence 714 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 702 may comprise a unique molecular identifying sequence 716 (e.g., unique molecular identifier (UMI)). In some cases, the unique molecular identifying sequence 716 may comprise from about 5 to about 8 nucleotides. Alternatively, the unique molecular identifying sequence 716 may compress less than about 5 or more than about 8 nucleotides. The unique molecular identifying sequence 716 may be a unique sequence that varies across individual nucleic acid molecules (e.g., 702, 718, 720, etc.) coupled to a single bead (e.g., bead 704). In some cases, the unique molecular identifying sequence 716 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 7 shows three nucleic acid molecules 702, 718, 720 coupled to the surface of the bead 704, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 708, 710, 712, etc.) and variable or unique sequence segments (e.g., 716) between different individual nucleic acid molecules coupled to the same bead.

A biological particle (e.g., cell, fixed cell, un-fixed cell, DNA, RNA, etc.) can be co-partitioned along with a barcode bearing bead 704. The barcoded nucleic acid molecules 702, 718, 720 can be released from the bead 704 in the partition. By way of example, in the context of analyzing sample RNA, the poly-T segment (e.g., 712) of one of the released nucleic acid molecules (e.g., 702) can hybridize to the poly-A tail of a mRNA molecule. Reverse transcription may result in a cDNA transcript of the mRNA, but which transcript includes each of the sequence segments 708, 710, 716 of the nucleic acid molecule 702. Because the nucleic acid molecule 702 comprises an anchoring sequence 714, it will more likely hybridize to and prime reverse transcription at the sequence end of the poly-A tail of the mRNA. Within any given partition, all of the cDNA transcripts of the individual mRNA molecules may include a common barcode sequence segment 710.

However, the transcripts made from the different mRNA molecules within a given partition may vary at the unique molecular identifying sequence 712 segment (e.g., UMI segment). Beneficially, even following any subsequent amplification of the contents of a given partition, the number of different UMIs can be indicative of the quantity of mRNA originating from a given partition, and thus from the biological particle (e.g., a cell, a fixed cell, an un-fixed cell, etc.). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing). The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

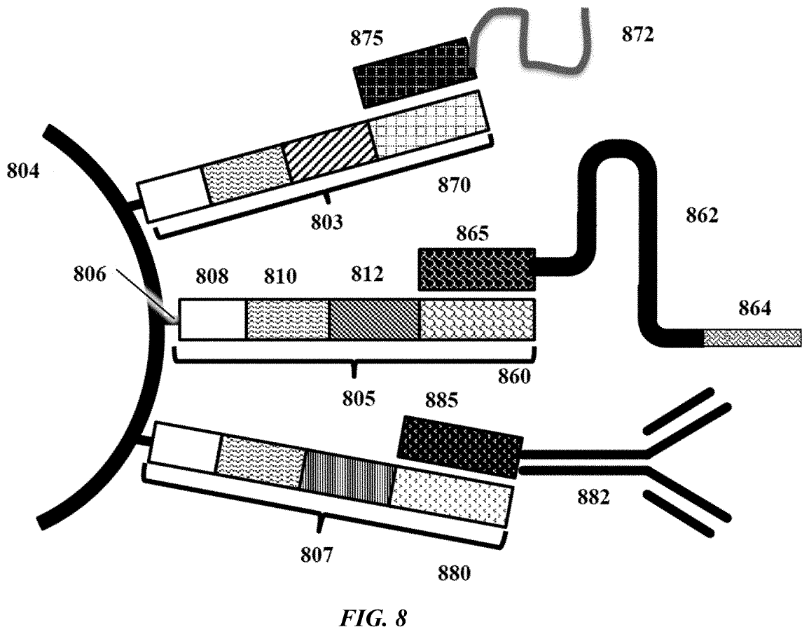

FIG. 8 illustrates another example of a barcode carrying bead. A nucleic acid molecule 805, such as an oligonucleotide, can be coupled to a bead 804 by a releasable linkage 806, such as, for example, a disulfide linker. The nucleic acid molecule 805 may comprise a first capture sequence 860. The same bead 804 may be coupled (e.g., via releasable linkage) to one or more other nucleic acid molecules 803, 807 comprising other capture sequences. The nucleic acid molecule 805 may be or comprise a barcode. As noted elsewhere herein, the structure of the barcode may comprise a number of sequence elements, such as a functional sequence 808 (e.g., flow cell attachment sequence, sequencing primer sequence, etc.), a barcode sequence 810 (e.g., bead-specific sequence common to bead, partition-specific sequence common to partition, etc.), and a unique molecular identifier 812 (e.g., unique sequence within different molecules attached to the bead), or partial sequences thereof. The capture sequence 860 may be configured to attach to a corresponding capture sequence 865. In some instances, the corresponding capture sequence 865 may be coupled to another molecule that may be an analyte or an intermediary carrier. For example, as illustrated in FIG. 8, the corresponding capture sequence 865 is coupled to a guide RNA molecule 862 comprising a target sequence 864, wherein the target sequence 864 is configured to attach to the analyte. Another oligonucleotide molecule 807 attached to the bead 804 comprises a second capture sequence 880 which is configured to attach to a second corresponding capture sequence 885. As illustrated in FIG. 8, the second corresponding capture sequence 885 is coupled to an antibody 882. In some cases, the antibody 882 may have binding specificity to an analyte (e.g., surface protein). Alternatively, the antibody 882 may not have binding specificity. Another oligonucleotide molecule 803 attached to the bead 804 comprises a third capture sequence 870 which is configured to attach to a second corresponding capture sequence 875. As illustrated in FIG. 8, the third corresponding capture sequence 875 is coupled to a molecule 872. The molecule 872 may or may not be configured to target an analyte. The other oligonucleotide molecules 803, 807 may comprise the other sequences (e.g., functional sequence, barcode sequence, UMI, etc.) described with respect to oligonucleotide molecule 805. While a single oligonucleotide molecule comprising each capture sequence is illustrated in FIG. 8, it will be appreciated that, for each capture sequence, the bead may comprise a set of one or more oligonucleotide molecules each comprising the capture sequence. For example, the bead may comprise any number of sets of one or more different capture sequences. Alternatively, or in addition, the bead 804 may comprise other capture sequences. Alternatively, or in addition, the bead 804 may comprise fewer types of capture sequences (e.g., two capture sequences). Alternatively or in addition, the bead 804 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

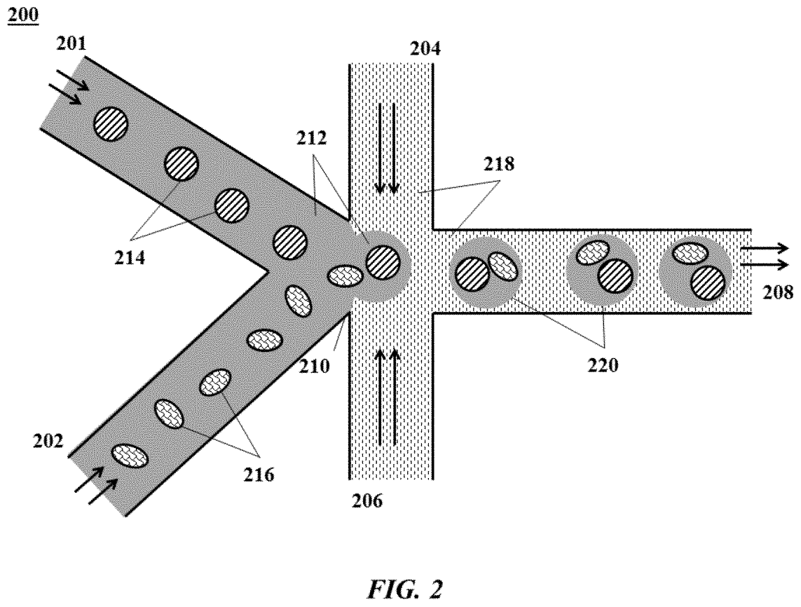

FIG. 2 shows an exemplary microfluidic channel structure 200 for generating discrete droplets encapsulating a barcode carrying bead 214 along with a biological particle 216. The channel structure 200 includes channel segments 201, 202, 204, 206 and 208 in fluid communication at a channel junction 210. In operation, the channel segment 201 transports an aqueous fluid 212 that can include a plurality of beads 214 (e.g., gel beads carrying barcode oligonucleotides) along the channel segment 201 into junction 210. The plurality of beads 214 may be sourced from a suspension of beads. For example, the channel segment 201 can be connected to a reservoir comprising an aqueous suspension of beads 214. The channel segment 202 transports the aqueous fluid 212 that includes a plurality of biological particles from a biological sample 216 along the channel segment 202 into junction 210. The plurality of biological particles 216 may be sourced from a suspension of biological sample. For example, the channel segment 202 may be connected to a reservoir comprising an aqueous suspension of biological particles 216. In some instances, the aqueous fluid 212 in either the first channel segment 201 or the second channel segment 202, or in both segments, can include one or more reagents, as further described elsewhere herein. For example, in some embodiments of the present disclosure, where the biological particles are from a fixed biological sample, the aqueous fluid in the first and/or second channel segments that delivers the biological sample and beads, respectively, can include an un-fixing agent. The second fluid 218 that is immiscible with the aqueous fluid 212 is delivered to the junction 210 from each of channel segments 204 and 206. Upon meeting of the aqueous fluid 212 from each of channel segments 201 and 202 and the second fluid 218 (e.g., a fluorinated oil) from each of channel segments 204 and 206 at the channel junction 210, the aqueous fluid 212 is partitioned into discrete droplets 220 in the second fluid 218 and flow away from the junction 210 along channel segment 208. The channel segment 208 can then deliver the discrete droplets encapsulating the biological particle and barcode carrying bead to an outlet reservoir fluidly coupled to the channel segment 208, where they can be collected.

As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological particles in an alternating fashion, such that, for example, a droplet comprises a single bead and a single biological particle.

Using such a channel system as exemplified in FIG. 2, discrete droplets 220 can be generated that encapsulate an individual particle of a biological sample, and one bead, wherein the bead can carry a barcode and/or another reagent. It is also contemplated, that in some instances, a discrete droplet may be generated using the channel system of FIG. 2, wherein droplet includes more than one individual biological particle or includes no biological sample. Similarly, in some embodiments, the discrete droplet may include more than one bead or no bead. A discrete droplet also may be completely unoccupied (e.g., no bead or biological sample).

In some embodiments, it is desired that the beads, biological particles from a biological sample, and generated discrete droplets flow along channels at substantially regular flow rates that generate a discrete droplet containing a single bead and a single biological particle. Regular flow rates and devices that may be used to provide such regular flow rates are known in the art, see e.g., U.S. Patent Publication No. 2015/0292988, which is hereby incorporated by reference herein in its entirety. In some embodiments, the flow rates are set to provide discrete droplets containing a single bead and a biological particle with a yield rate of greater than 5%, 10%, 20%, 30%, 40%, 50%, 60%, 70%, 80%, 90%, or 95%.

### G. Supports

Supports, such as beads, that can carry barcodes and/or other reagents are useful with the methods of the present disclosure and can include, without limitation, supports that are porous, non-porous, solid, semi-solid, semi-fluidic, fluidic, and/or a combination thereof. In some embodiments, the support is a bead that is made of a material that is dissolvable, disruptable, and/or degradable, such as a gel bead comprising a hydrogel. Alternatively, in some embodiments, the support is not degradable.

In some embodiments of the present disclosure, the support is a bead that can be encapsulated in a discrete droplet with a biological sample. Typically, the bead useful in the embodiments disclosed herein comprise a hydrogel. Such gel beads can be formed from molecular precursors, such as a polymeric or monomeric species, that undergo a reaction to form crosslinked gel polymer. Another semi-solid bead useful in the present disclosure is a liposomal bead. In some embodiments, beads used can be solid beads that comprise a metal including iron oxide, gold, and silver. In some cases, the bead may be a silica bead. In some cases, the bead can be rigid. In other cases, the bead may be flexible and/or compressible. Generally, the beads can be of any suitable shape. Examples of bead shapes include, but are not limited to, spherical, non-spherical, oval, oblong, amorphous, circular, cylindrical, and variations thereof.

In some embodiments, a plurality or population of beads can be used. The plurality of beads used in the embodiments can be of uniform size, having a relatively monodisperse size distribution, or they can comprise a collection of heterogeneous sizes. In some cases, the diameter of a bead is at least about 1 micron (μm), 5 μm, 10 μm, 20 μm, 30 μm, 40 μm, 50 μm, 60 μm, 70 μm, 80 μm, 90 μm, 100 μm, 250 μm, 500 μm, 1000 μm (1 mm), or greater. In some cases, a bead may have a diameter of less than about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

Typically, where it is desirable to provide a consistent amount of a reagent within a discrete droplet, the use of relatively consistent bead characteristics, such as size, provides overall consistency in the content of each droplet. For example, the beads useful in the embodiments of the present disclosure can have size distributions that have a coefficient of variation in their cross-sectional dimensions of less than 50%, less than 40%, less than 30%, less than 20%, and in some cases less than 15%, less than 10%, less than 5%, or less.

The beads useful in the methods of the present disclosure can comprise a range of natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

Although FIG. 1 and FIG. 2 have been described in terms of providing substantially singly occupied discrete droplets, it is also contemplated in certain embodiments that it is desirable to provide multiply occupied discrete droplets, e.g., a single droplet that contains two, three, four or more cells from a biological sample, and/or multiple different beads, such as a bead carrying a barcode nucleic acid molecule and/or a support (e.g., a bead) carrying a reagent such as an un-fixing agent or assay reagent. Accordingly, as noted elsewhere herein, the flow characteristics of the biological particle and/or the beads can be controlled to provide for such multiply occupied droplets. In particular, the flow parameters of the liquids used in the channel structures may be controlled to provide a given droplet occupancy rate greater than about 50%, greater than about 75%, and in some cases greater than about 80%, 90%, 95%, or higher.

In some embodiments, the beads useful in the methods of the present disclosure are supports (e.g., beads) capable of delivering reagents (e.g., an un-fixing agent, and/or an assay reagent) into the discrete droplet generated containing the biological particle. In some embodiments, the different beads (e.g., containing different reagents) can be introduced from different sources into different inlets leading to a common droplet generation junction (e.g., junction 210). In such cases, the flow and frequency of the different beads into the channel or junction may be controlled to provide for a certain ratio of supports from each source, while ensuring a given pairing or combination of such supports (e.g., beads) into a partition with a given number of biological particles (e.g., one biological particle and one bead per partition).

The discrete droplets described herein generally comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less. In some embodiments, the discrete droplets generated that encapsulate a biological particle have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. It will be appreciated that the sample fluid volume, e.g., including co-partitioned biological particles and/or beads, within the droplets may be less than about 90% of the above described volumes, less than about 80%, less than about 70%, less than about 60%, less than about 50%, less than about 40%, less than about 30%, less than about 20%, or less than about 10% of the above described volumes.

The methods of generating discrete droplets useful with the methods of the present disclosure, result in the generation of a population or plurality of discrete droplets containing a biological particle (e.g., a biological particle from a fixed biological sample) and other reagents (e.g., an un-fixing agent). Generally, the methods are easily controlled to provide for any suitable number of droplets. For example, at least about 1,000 discrete droplets, at least about 5,000 discrete droplets, at least about 10,000 discrete droplets, at least about 50,000 discrete droplets, at least about 100,000 discrete droplets, at least about 500,000 discrete droplets, at least about 1,000,000 discrete droplets, at least about 5,000,000 discrete droplets, at least about 10,000,000 discrete droplets, or more discrete droplets can be generated or otherwise provided. Moreover, the plurality of discrete droplets may comprise both unoccupied and occupied droplets.

As described elsewhere herein, in some embodiments of the methods of the present disclosure, the generated discrete droplets encapsulating a biological particle, and optionally, one or more different beads, also contain other reagents. In some embodiments, the other reagents encapsulated in the droplet include lysis and/or un-fixing agents that act to release and/or un-fix the biomolecule contents of the biological particle within the droplet. In some embodiments, the lysis and/or un-fixing agents can be contacted with the biological sample suspension concurrently with, or immediately prior to, the introduction of the biological particles into the droplet generation junction of the microfluidic system (e.g., junction 210). In some embodiments, the agents are introduced through an additional channel or channels upstream of the channel junction.

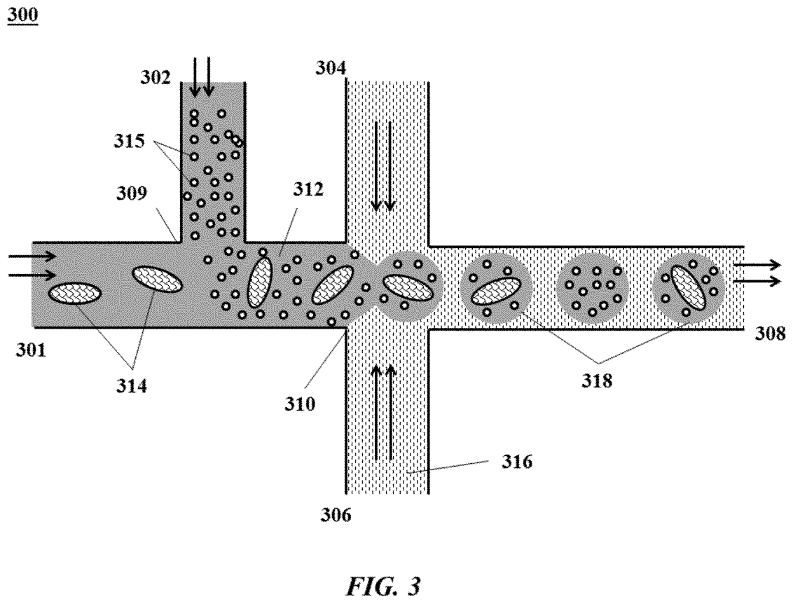

In some embodiments, a biological particle can be co-partitioned along with the other reagents. FIG. 3 shows an example of a microfluidic channel structure 300 for co-partitioning biological particles and other reagents, including lysis and/or un-fixing agents. The channel structure 300 can include channel segments 301, 302, 304, 306 and 308. Channel segments 301 and 302 communicate at a first channel junction 309. Channel segments 302, 304, 306, and 308 communicate at a second channel junction 310. In exemplary co-partitioning operation, the channel segment 301 may transport an aqueous fluid 312 that includes a plurality of biological particles 314 (e.g., a fixed biological sample) along the channel segment 301 into the second junction 310. As an alternative or in addition to, channel segment 301 may transport beads (e.g., beads that carry barcodes). For example, the channel segment 301 may be connected to a reservoir comprising an aqueous suspension of biological particles 314. Upstream of, and immediately prior to reaching, the second junction 310, the channel segment 301 may meet the channel segment 302 at the first junction 309. The channel segment 302 can transport a plurality of reagents 315 (e.g., lysis or un-fixing agents) in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological particles 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., a fluorinated oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 is partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be collected for further analysis.

Discrete droplets generated can include an individual biological particle 314 and/or one or more reagents 315, depending on what reagents are included in channel segment 302. In some instances, a discrete droplet generated may also include a barcode carrying bead (not shown), such as can be added via other channel structures described elsewhere herein. In some instances, a discrete droplet may be unoccupied (e.g., no reagents, no biological particles). Generally, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electro-kinetic pumping, vacuum, capillary or gravity flow, or the like.

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w, ho, a, etc.) of the channel structure 400. A plurality of droplets can be collected in the reservoir 404 by continuously injecting the aqueous fluid 408 from the channel segment 402 through the junction 406.

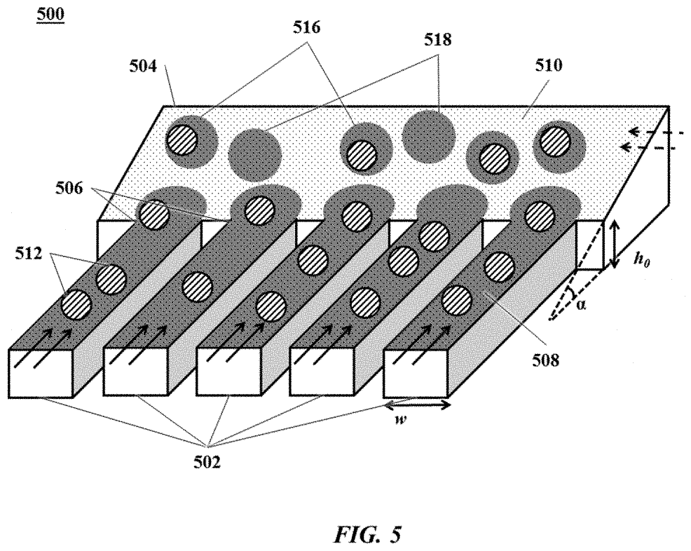

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

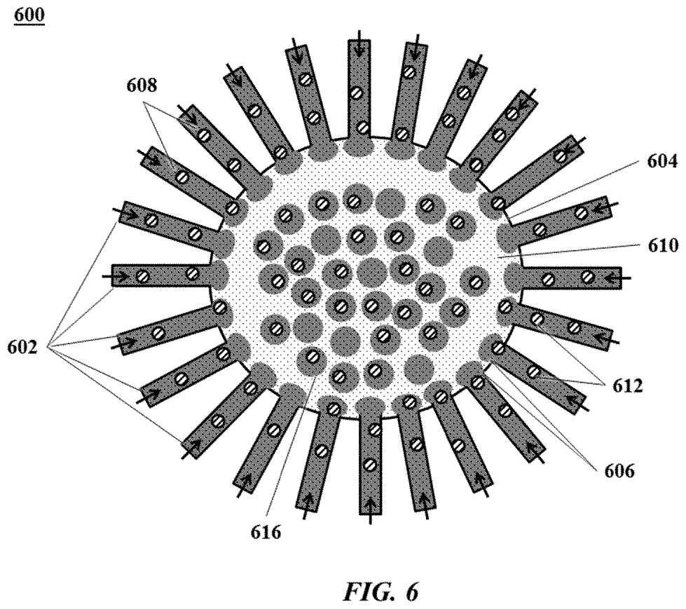

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 4 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof. Additional aspects of the microfluidic structures depicted in FIGS. 4-6, including systems and methods implementing the same, are provided in US Published Patent Application No 20190323088, which is incorporated herein by reference in its entirety.

Once the lysis and/or un-fixing agents are co-partitioned in a droplet with a fixed biological particle, these reagents can facilitate the release and un-fixing of the biomolecular contents of the biological particle within the droplet. As described elsewhere herein, the un-fixed biomolecular contents released in a droplet remain discrete from the contents of other droplets, thereby allowing for detection and quantitation of the biomolecular analytes of interest present in that distinct biological sample.

Examples of lysis agents useful in the methods of the present disclosure include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the biological particles to cause the release of the biological samples' contents into the droplet. For example, in some cases, surfactant-based lysis solutions may be used to lyse cells, although these may be less desirable for emulsion based systems where the surfactants can interfere with stable emulsions. In some embodiment, the lysis solutions can include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

In addition to the lysis and/or un-fixing agents co-partitioned into discrete droplets with the biological particles, it is further contemplated that other assay reagents can also be co-partitioned in the droplet. For example, DNase and RNase inactivating agents or inhibitors, such as proteinase K, chelating agents, such as EDTA, proteases, such as subtilisin A, and other reagents employed in removing or otherwise reducing negative activity or impact of different cell lysate components on subsequent processing of nucleic acids.

In some embodiments, the biological particles from a biological sample are provided in or encapsulated in discrete partitions (e.g., wells or droplets) with other reagents are exposed to an appropriate stimulus to release the biomolecular contents of the sample particles and/or the contents of a co-partitioned support (e.g., a bead). For example, in some embodiments, a chemical stimulus may be co-partitioned in the droplet along with a biological particle and a support (e.g., a bead such as a gel bead) to allow for the degradation of the support and release of the its contents into the droplet. In some embodiments, a discrete droplet can be generated with a fixed biological particle and an un-fixing agent, wherein the un-fixing agent is contained in a support (e.g., a bead) that can be degraded by heat stimulus. In such an embodiment, the droplet is exposed to heat stimulus thereby degrading the bead and releasing the un-fixing agent. In another embodiment, it is contemplated that a droplet encapsulating a fixed biological particle from a fixed biological sample, and two different beads (e.g., one bead carrying an un-fixing agent, and one bead carrying assay reagents), wherein the contents of the two different beads are released by non-overlapping stimuli (e.g., a chemical stimulus and a heat stimulus). Such an embodiment can allow the release of the different reagents into the same discrete droplet at different times. For example, a first bead, triggered by heat stimulus, releases an un-fixing agent into the droplet, and then after a set time, a second bead, triggered by a chemical stimulus, releases assay reagents that detect analytes of the un-fixed biological particle.

Additional assay reagents may also be co-partitioned into discrete droplets with the biological samples, such as endonucleases to fragment a biological sample's DNA, DNA polymerase enzymes and dNTPs used to amplify the biological sample's nucleic acid fragments and to attach the barcode molecular tags to the amplified fragments. Other enzymes may be co-partitioned, including without limitation, polymerase, transposase, ligase, proteinase K, DNase, subtilisin A, etc. Additional assay reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some embodiments, template switching can be used to increase the length of cDNA generated in an assay. In some embodiments, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner.

Once the contents of a biological sample cell are released into a discrete droplet, the biomolecular components (e.g., macromolecular constituents of biological samples, such as RNA, DNA, or proteins) contained therein may be further processed within the droplet. In accordance with the methods and systems described herein, the biomolecular contents of individual biological samples can be provided with unique barcode identifiers, and upon characterization of the biomolecular components (e.g., in a sequencing assay) they may be attributed as having been derived from the same biological sample. The ability to attribute characteristics to individual biological samples or groups of biological samples is provided by the assignment of a nucleic acid barcode sequence specifically to an individual biological sample or groups of biological samples.

In some aspects, the unique identifier barcodes are provided in the form of nucleic acid molecules (e.g., oligonucleotides) that comprise sequences that may be attached to or otherwise associated with the nucleic acid contents of individual biological sample, or to other components of the biological sample, and particularly to fragments of those nucleic acids. In some embodiments, only one nucleic acid barcode sequence is associated with a given discrete droplet, although in some cases, two or more different barcode sequences may be present. The nucleic acid barcode sequences can include from about 6 to about 20 or more nucleotides within the sequence of the nucleic acid molecules (e.g., oligonucleotides). In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

In some embodiments, the nucleic acid barcode molecules can also comprise other functional sequences useful in the processing of the nucleic acids from the biological sample in the droplet. These functional sequences can include, e.g., targeted or random/universal amplification primer sequences for amplifying the nucleic acid molecules from the individual biological samples within the partitions while attaching the associated barcode sequences, sequencing primers or primer recognition sites, hybridization or probing sequences, e.g., for identification of presence of the sequences or for pulling down barcoded nucleic acid molecules, or any of a number of other potential functional sequences.

In some embodiments, large numbers of nucleic acid barcode molecules (e.g., oligonucleotides) are releasably attached to beads, wherein all of the nucleic acid molecules attached to a particular bead will include the same nucleic acid barcode sequence, but where a large number of diverse barcode sequences are represented across the population of beads used. In some embodiments, gel beads (e.g., comprising polyacrylamide polymer matrices), are used as a solid support and delivery vehicle for the nucleic acid molecules into the droplets, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more.

The nucleic acid barcode molecules can be released from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological samples and may be degraded for release of the attached nucleic acid molecules through exposure to a reducing agent, such as DTT.

### H. Cold Protease Treatment of Fixed Biological Samples in Partition-Based Assays

As disclosed elsewhere herein, the methods of the present disclose allow a fixed, stabilized, biological sample (e.g., formaldehyde-fixed biopsy cells) to be provided in a discrete partition (e.g., encapsulated in a droplet), optionally, as a single cell, together with a low-temperature active protease, and optionally, an un-fixing agent that is capable of reversing the fixation. The protease and un-fixing agent can act to release and un-fix the cellular analytes within the sample (e.g., cell, cells, tissue sample, or other type of biological sample), thereby allowing the cellular analytes of the sample to be assayed as if they were obtained from a fresh sample. Further, the methods allow for a fresh biological sample to be collected, immediately fixed (e.g., with formaldehyde), and then stored for a period of time before it is subjected to the low-temperature protease treatment and an un-fixing agent. Accordingly, it is contemplated that the methods of the present disclosure can be carried out wherein the amount of time prior to generating the discrete droplet when the biological sample is fixed is at least 1 hour, at least 2 hours, at least 6 hours, at least 12 hours, at least 24 hours, at least 1 week, at least 1 month, at least 6 months, or longer.

The present disclosure also provides an assay method that comprises the steps of: (a) generating a discrete droplet encapsulating a fixed biological sample, a low-temperature active protease, an un-fixing agent, and assay reagents; and (b) detecting analytes from the reaction of the assay reagents and the un-fixed biological sample. Optionally, the steps of the method can further comprise fixing the biological sample prior to generating the discrete droplet.

A wide range of partition-based assays and systems are known in the art. Assays and systems that are suitable for use with the present disclosure include, without limitation, those described in US Patent Nos. 9694361, 10357771, 10273541, and 10011872, as well as US Published Patent Application Nos. 20180105808, 20190367982, and 20190338353, each of which is incorporated herein by reference in its entirety. It is contemplated that any assay that can be carried out using a fresh biological sample, such as a single cell encapsulated in a droplet with a bead carrying a barcode, can also be carried out using a fixed biological sample, the unfixing agents as disclosed herein, and the methods of the present disclosure. That is, the in any partition-based assay the fresh biological sample can be fixed prior to running the assay protocol, and the fixed biological sample used. In such an assay the protocol comprises encapsulating the fixed biological together with an un-fixing agent and assay reagents in a discrete droplet.

Exemplary assays include single-cell transcription profiling, single-cell sequence analysis, immune profiling of individual T and B cells, single-cell chromatin accessibility analysis (e.g., ATAC seq analysis). These exemplary assays can be carried out using commercially available systems for encapsulating biological samples, gel beads, barcodes, and/or other compounds/materials in droplets, such as The Chromium System (10X Genomics, Pleasanton, Calif., USA).

In some embodiments of the assay methods, the discrete droplet further comprises one or more beads. In some embodiments, the bead(s) can contain the assay reagents and/or the un-fixing agent. In some embodiments, a barcode is carried by or contained in a bead. Compositions, methods and systems for sample preparation, amplification, and sequencing of biomolecules from single cells encapsulated with barcodes in droplets are provided in e.g., US Pat. Publication No. 20180216162A1, which is hereby incorporated by reference herein.

Assay reagents can include those used to perform one or more additional chemical or biochemical operations on a biological sample encapsulated in a droplet. Accordingly, assay reagents useful in the assay method include any reagents useful in performing a reaction such as nucleic acid modification (e.g., ligation, digestion, methylation, random mutagenesis, bisulfite conversion, uracil hydrolysis, nucleic acid repair, capping, or decapping), nucleic acid amplification (e.g., isothermal amplification or PCR), nucleic acid insertion or cleavage (e.g., via CRISPR/Cas9-mediated or transposon-mediated insertion or cleavage), and/or reverse transcription. Additionally, useful assay reagents can include those that allow the preparation of a target sequence or sequencing reads that are specific to the macromolecular constituents of interest at a higher rate than to non-target sequence specific reads.

In addition, the present disclosure provides compositions and systems related to the analysis of biological samples prepared with the methods. In one embodiment, the present disclosure provides a composition comprising a plurality of partitions, wherein a subset of said plurality of partitions comprises fixed cells, a low-temperature active protease, and optionally, an un-fixing agent. In one other embodiment, the subset of partitions further comprises a protease. In another embodiment, a partition of the plurality of partitions comprises a fixed cell, low-temperature active protease, and an un-fixing agent. In certain embodiments, the fixed cell is a single fixed cell. In other embodiments the present disclosure provides a composition comprising a partition, wherein the partition comprises a fixed cell, a low-temperature active protease, and an un-fixing agent, as described herein. The partition may be a droplet or a well.

In some embodiments, the partition or partitions described herein may further comprise one or more of the following: a reverse transcriptase (RT), a bead, and reagents for a nucleic acid extension reaction. In at least one embodiment, the protease and/or un-fixing agent compositions can be provided at a temperature other than ambient temperature. In one embodiment, the temperature is below ambient temperature or above ambient temperature.

As described elsewhere herein, partitioning approaches may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions. For example, an occupied partition according the present disclosure comprises a fixed cell, a low-temperature active protease composition, and an un-fixing agent.

In another aspect, the present disclosure concerns methods for the partitioning of a plurality of fixed cells into individual partitions. In some cases, about 10, about 20, about 30, about 40, about 50, about 60, about 70, about 80, about 90, about 100, about 200, about 300, about 400, about 500, about 600, about 700, about 800, about 900, about 1000, about 2000, about 3000, about 4000, about 5000, about 6000, about 7000, about 8000, about 9000, about 10,000, about 15,000, about 20,000, about 25,000, about 30,000, about 35,000, about 40,000, about 50,000, about 60,000, about 70,000, about 80,000, about 90,000 or about 100,000 fixed cells may be partitioned into individual partitions. In some instances, the method further comprises partitioning about 50 to about 20,000 fixed cells with each of a plurality of supports comprising the adaptor comprising the barcode sequence, wherein the barcode sequence is unique among each of the plurality of supports.

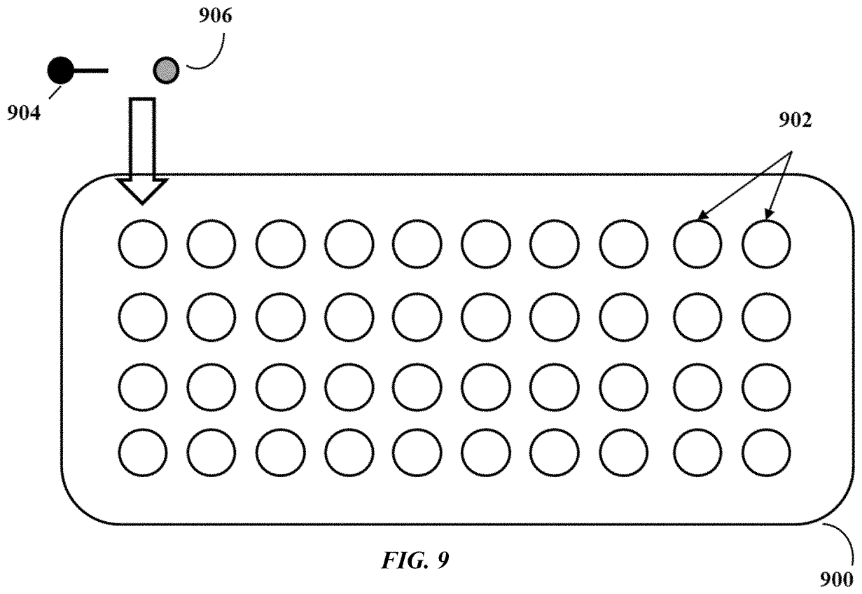

FIG. 9 schematically illustrates an example of a microwell array. The array can be contained within a substrate 900. The substrate 900 comprises a plurality of wells 902. The wells 902 may be of any size or shape, and the spacing between the wells, the number of wells per substrate, as well as the density of the wells on the substrate 900 can be modified, depending on the particular application. In one such example application, a sample molecule 906, which may comprise a cell (e.g., a fixed cell or an un-fixed cell) or cellular components (e.g., nucleic acid molecules) is co-partitioned with a bead 904, which may comprise a nucleic acid barcode molecule coupled thereto. The wells 902 may be loaded using gravity or other loading technique (e.g., centrifugation, liquid handler, acoustic loading, optoelectronic, etc.). In some instances, at least one of the wells 902 contains a single sample molecule 906 (e.g., cell) and a single bead 904.

Reagents may be loaded into a well either sequentially or concurrently. In some cases, reagents are introduced to the device either before or after a particular operation. In some cases, reagents (which may be provided, in certain instances, in droplets or beads) are introduced sequentially such that different reactions or operations occur at different steps. The reagents (or droplets or beads) may also be loaded at operations interspersed with a reaction or operation step. For example, droplets or beads comprising reagents for fragmenting polynucleotides (e.g., restriction enzymes) and/or other enzymes (e.g., transposases, ligases, polymerases, etc.) may be loaded into the well or plurality of wells, followed by loading of droplets or beads comprising reagents for attaching nucleic acid barcode molecules to a sample nucleic acid molecule. Reagents may be provided concurrently or sequentially with a sample, such as a cell (e.g., a fixed cell or an un-fixed cell) or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead or droplet. These beads or droplets may be loaded into a partition (e.g., a microwell) before, after, or concurrently with the loading of a cell (e.g., a fixed cell or an un-fixed cell), such that each cell is contacted with a different bead or droplet. This technique may be used to attach a unique nucleic acid barcode molecule to nucleic acid molecules obtained from each cell (e.g., a fixed cell or an un-fixed cell). Alternatively or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell, e.g., a single fixed cell or a single un-fixed cell) and a single bead (such as those described herein, which may, in some instances, also be provided or encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where cells, e.g., fixed cells or un-fixed cells are loaded in the microwells, one of the droplets may comprise reagents for further processing, e.g., lysis reagents for lysing the cell, upon droplet merging.

A droplet may be partitioned into a well. The droplets may be selected or subjected to pre-processing prior to loading into a well. For instance, the droplets may comprise cells, e.g., fixed cells or un-fixed cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell (e.g., a fixed cell or an un-fixed cell) distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. For instance, the nucleic acid barcode molecules may comprise a chemical cross-linker which may be cleaved upon application of a stimulus (e.g., photo-, magnetic, chemical, biological, stimulus). The released nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell or cellular components) or derivatives thereof. Characterization techniques such as microscopy or imaging may be useful in measuring sample profiles in fixed spatial locations. For instance, when cells (e.g., fixed cells or un-fixed cells) are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, cell-cell interactions (when two or more cells are co-partitioned). Alternatively or in addition to, imaging may be used to characterize a quantity of amplification products in the well.

In operation, a well may be loaded with a sample and reagents, simultaneously or sequentially. When cells (e.g., fixed cells or un-fixed cells) are loaded, the well may be subjected to washing, e.g., to remove excess cells from the well, microwell array, or plate. Similarly, washing may be performed to remove excess beads or other reagents from the well, microwell array, or plate. In addition, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

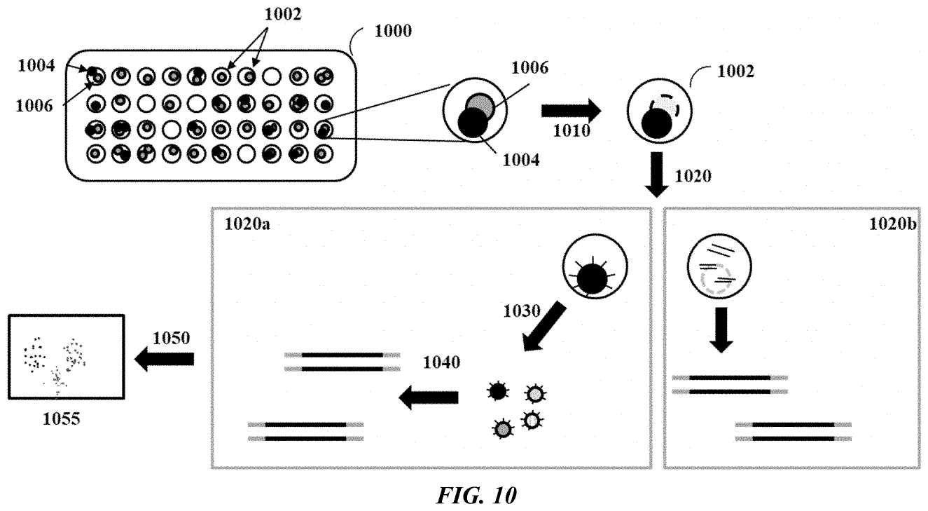

FIG. 10 schematically shows an example workflow for processing nucleic acid molecules within a sample. A substrate 1000 comprising a plurality of microwells 1002 may be provided. A sample 1006 which may comprise a cell (e.g., a fixed cell or an un-fixed cell), cellular components or analytes (e.g., proteins and/or nucleic acid molecules) can be co-partitioned, in a plurality of microwells 1002, with a plurality of beads 1004 comprising nucleic acid barcode molecules. During process 1010, the sample 1006 may be processed within the partition. For instance, the cell may be subjected to conditions sufficient to lyse the cells (e.g., fixed cells or un-fixed cells) and release the analytes contained therein. In process 1020, the bead 1004 may be further processed. By way of example, processes 1020a and 1020b schematically illustrate different workflows, depending on the properties of the bead 1004.

In 1020a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 1030, the beads 1004 from multiple wells 1002 may be collected and pooled. Further processing may be performed in process 1040. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1050, further characterization, such as sequencing may be performed to generate sequencing reads. The sequencing reads may yield information on individual cells or populations of cells (e.g., fixed cells or un-fixed cells), which may be represented visually or graphically, e.g., in a plot 1055.

In 1020b, the bead comprises nucleic acid barcode molecules that are releasably attached thereto, as described below. The bead may degrade or otherwise release the nucleic acid barcode molecules into the well 1002; the nucleic acid barcode molecules may then be used to barcode nucleic acid molecules within the well 1002. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1050, further characterization, such as sequencing may be performed to generate sequencing reads. The sequencing reads may yield information on individual cells or populations of cells (e.g., fixed cells or un-fixed cells), which may be represented visually or graphically, e.g., in a plot 1055

In 1020b, the bead comprises nucleic acid barcode molecules that are releasably attached thereto, as described below. The bead may degrade or otherwise release the nucleic acid barcode molecules into the well 1002; the nucleic acid barcode molecules may then be used to barcode nucleic acid molecules within the well 1002. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1050, further characterization, such as sequencing may be performed to generate sequencing reads. The sequencing reads may yield information on individual cells or populations of cells (e.g., fixed cells or un-fixed cells), which may be represented visually or graphically, e.g., in a plot 1055.

### I. Additional Methods

The present disclosure provides methods and systems for multiplexing, and otherwise increasing throughput of samples (e.g., cells, fixed cells or un-fixed cells) for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cells (e.g., cells, fixed cells or un-fixed cells) or cell features may be used to characterize cells and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence (e.g., a reporter sequence) that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

In a particular example, a library of potential cell feature labelling agents may be provided, where the respective cell feature labelling agents are associated with nucleic acid reporter molecules, such that a different reporter oligonucleotide sequence is associated with each labelling agent capable of binding to a specific cell feature. In other aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

For workflows comprising the use of fixation agents and/or un-fixing agents, labelling agents may be used to label samples (e.g., cells, fixed cells or un-fixed cells) at different points in time. In one embodiment, a plurality of cells is labeled prior to treatment with a fixation agent and/or after treatment with a fixation agent. In another embodiment, a plurality of fixed cells is labeled prior to treatment with an un-fixing agent and/or after treatment with an un-fixing agent. In one additional embodiment, a plurality of un-fixed cells is labeled prior to partitioning into partitions (e.g., wells or droplets) for further processing. In another embodiment, the methods, compositions, systems, and kits described herein provide labeled cells, labeled fixed cells or labeled un-fixed cells.

Labelling agents capable of binding to or otherwise coupling to one or more cells may be used to characterize a cell as belonging to a particular set of cells. For example, labeling agents may be used to label a sample of cells or a group of cells. In this way, a group of cells may be labeled as different from another group of cells. In an example, a first group of cells may originate from a first sample and a second group of cells may originate from a second sample. Labelling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling a cell may comprise subjecting the antibody-linked barcode molecule or the epitope binding fragment-linked barcode molecule to conditions suitable for binding the antibody to a molecule present on a surface of the cell. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension A dissociation constant (Kd) between the antibody or an epitope binding fragment thereof and the molecule to which it binds may be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM. 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 μM, 800 μM, 700 μM, 600 μM, 500 μM, 400 μM, 300 μM, 200 μM, 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, or 1 μM. For example, the dissociation constant may be less than about 10 μM.

In another example, a reporter oligonucleotide may be coupled to a cell-penetrating peptide (CPP), and labeling cells may comprise delivering the CPP coupled reporter oligonucleotide into an analyte carrier. Labeling analyte carriers may comprise delivering the CPP conjugated oligonucleotide into a cell and/or cell bead by the cell-penetrating peptide. A CPP that can be used in the methods provided herein can comprise at least one non-functional cysteine residue, which may be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP Cell-penetrating peptides useful in the methods provided herein can have the capability of inducing cell penetration for at least about 30%, 40%, 50%. 60%, 70%, 80%. 90%, 95%, 96%, 97%, 98%, 99%, or 100% of cells of a cell population. The CPP may be an arginine-rich peptide transporter. The CPP may be Penetratin or the Tat peptide. In another example, a reporter oligonucleotide may be coupled to a fluorophore or dye, and labeling cells may comprise subjecting the fluorophore-linked barcode molecule to conditions suitable for binding the fluorophore to the surface of the cell. In some instances, fluorophores can interact strongly with lipid bilayers and labeling cells may comprise subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2): e87649, which is hereby incorporated by reference in its entirety for all purposes, for a description of organic fluorophores.

A reporter oligonucleotide may be coupled to a lipophilic molecule, and labeling cells may comprise delivering the nucleic acid barcode molecule to a membrane of a cell or a nuclear membrane by the lipophilic molecule. Lipophilic molecules can associate with and/or insert into lipid membranes such as cell membranes and nuclear membranes In some cases, the insertion can be reversible. In some cases, the association between the lipophilic molecule and the cell or nuclear membrane may be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus. In one embodiment, a reporter oligonucleotide coupled to a lipophilic molecule will remain associated with and/or inserted into lipid membrane (as described herein) via the lipophilic molecule until lysis of the cell occurs, e.g., inside a partition.

A reporter oligonucleotide may be part of a nucleic acid molecule comprising any number of functional sequences, as described elsewhere herein, such as a target capture sequence, a random primer sequence, and the like, and coupled to another nucleic add molecule that is, or is derived from, the analyte.

Prior to partitioning, the cells may be incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

As described elsewhere herein, libraries of labelling agents may be associated with a particular cell feature as well as be used to identify analytes as originating from a particular cell population, or sample. Cell populations may be incubated with a plurality of libraries such that a cell or cells comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some instances, these reporter oligonucleotides may comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEGS-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide.

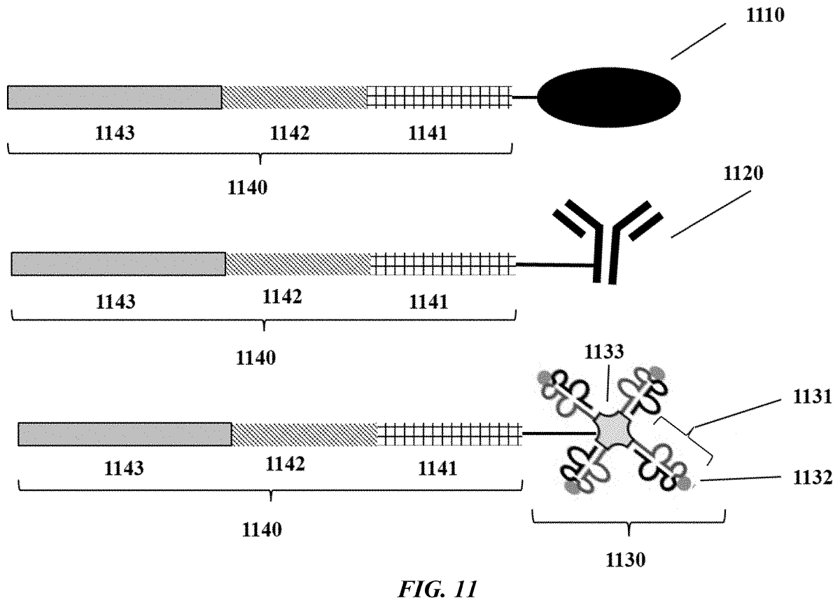

FIG. 11 describes exemplary labelling agents (1110, 1120, 1130) comprising reporter oligonucleotides (1140) attached thereto. Labelling agent 1110 (e.g., any of the labelling agents described herein) is attached (either directly, e.g., covalently attached, or indirectly) to reporter oligonucleotide 1140. Reporter oligonucleotide 1140 may comprise barcode sequence 1142 that identifies labelling agent 1110. Reporter oligonucleotide 1140 may also comprise one or more functional sequences 1143 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 11, in some instances, reporter oligonucleotide 1140 conjugated to a labelling agent (e.g., 1110, 1120, 1130) comprises a primer sequence 1141, a barcode sequence 1142 that identifies the labelling agent (e.g., 1110, 1120, 1130), and functional sequence 1143. Functional sequence 1143 may be configured to hybridize to a complementary sequence, such as a complementary sequence present on a nucleic acid barcode molecule 1190 (not shown), such as those described elsewhere herein. In some instances, nucleic acid barcode molecule 1190 is attached to a support (e.g., a bead, such as a gel bead), such as those described elsewhere herein. For example, nucleic acid barcode molecule 1190 may be attached to the support via a releasable linkage (e.g., comprising a labile bond), such as those described elsewhere herein. In some instances, reporter oligonucleotide 1140 comprises one or more additional functional sequences, such as those described above.

In some instances, the labelling agent 1110 is a protein or polypeptide (e.g., an antigen or prospective antigen) comprising reporter oligonucleotide 1140. Reporter oligonucleotide 1140 comprises barcode sequence 1142 that identifies polypeptide 1110 and can be used to infer the presence of an analyte, e.g., a binding partner of polypeptide 1110 (i.e., a molecule or compound to which polypeptide 1110 can bind). In some instances, the labelling agent 1110 is a lipophilic moiety (e.g., cholesterol) comprising reporter oligonucleotide 1140, where the lipophilic moiety is selected such that labelling agent 1110 integrates into a membrane of a cell or nucleus. Reporter oligonucleotide 1140 comprises barcode sequence 1142 that identifies lipophilic moiety 1110 which in some instances is used to tag cells (e.g., groups of cells, cell samples, etc.) and may be used for multiplex analyses as described elsewhere herein. In some instances, the labelling agent is an antibody 1120 (or an epitope binding fragment thereof) comprising reporter oligonucleotide 1140. Reporter oligonucleotide 1140 comprises barcode sequence 1142 that identifies antibody 1120 and can be used to infer the presence of, e.g., a target of antibody 1120 (i.e., a molecule or compound to which antibody 1120 binds). In other embodiments, labelling agent 1130 comprises an MHC molecule 1131 comprising peptide 1132 and reporter oligonucleotide 1140 that identifies peptide 1132. In some instances, the MHC molecule is coupled to a support 1133. In some instances, support 1133 may be a polypeptide, such as streptavidin, or a polysaccharide, such as dextran. In some instances, reporter oligonucleotide 1140 may be directly or indirectly coupled to MHC labelling agent 1130 in any suitable manner. For example, reporter oligonucleotide 1140 may be coupled to MHC molecule 1131, support 1133, or peptide 1132. In some embodiments, labelling agent 1130 comprises a plurality of MHC molecules, (e.g., is an MHC multimer, which may be coupled to a support (e.g., 1133)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969, each of which is herein entirely incorporated by reference for all purposes.

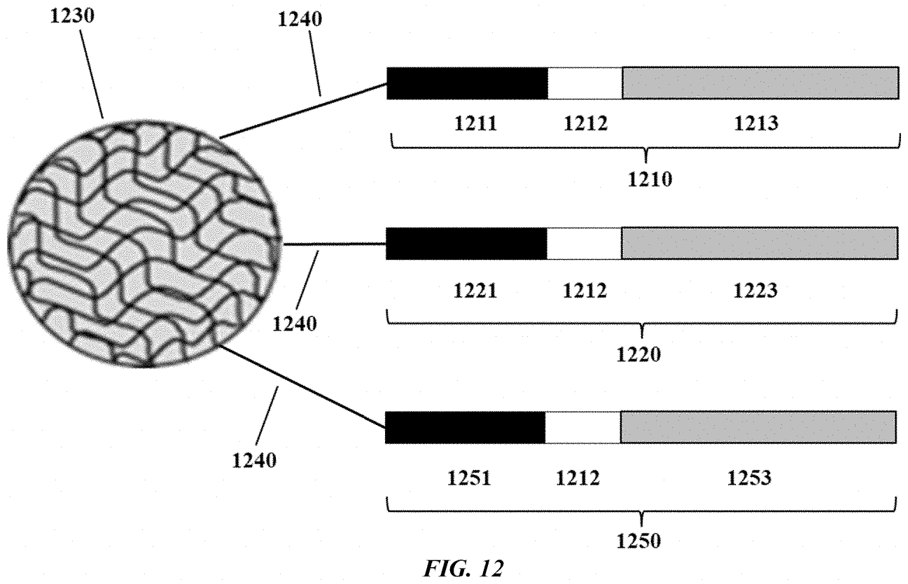

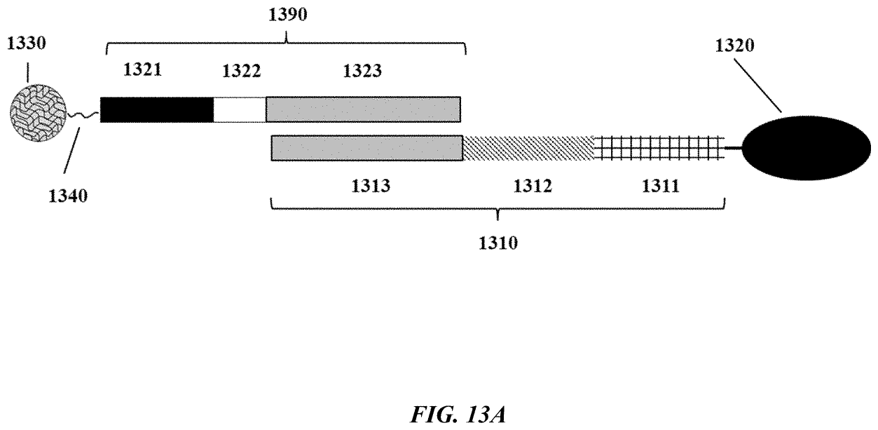

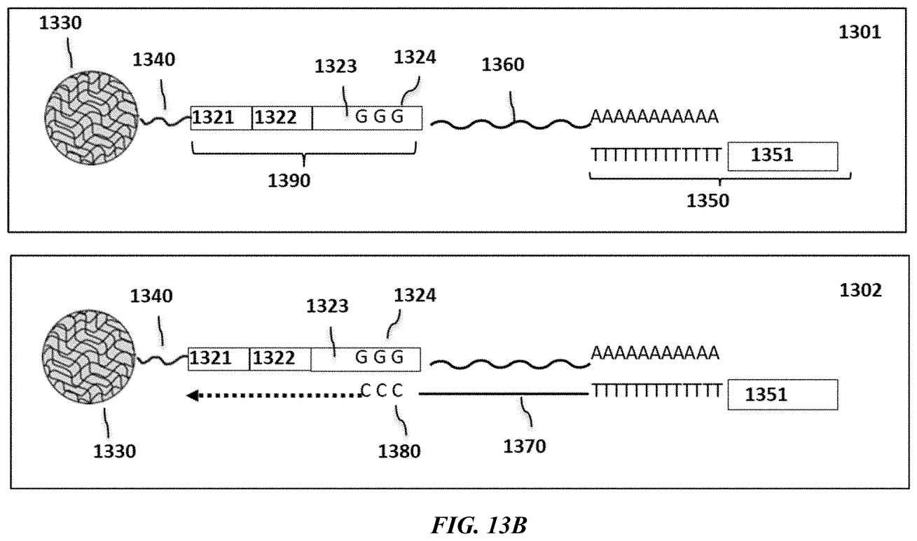

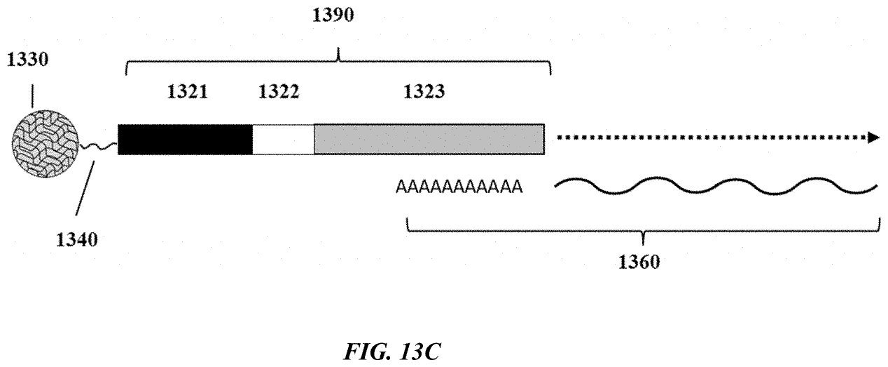

FIG. 12 illustrates another example of a barcode carrying bead. In some embodiments, analysis of multiple analytes (e.g., RNA and one or more analytes using labelling agents described herein) may comprise nucleic acid barcode molecules as generally depicted in FIG. 12. In some embodiments, nucleic acid barcode molecules 1210 and 1212 are attached to support 1230 via a releasable linkage 1240 (e.g., comprising a labile bond) as described elsewhere herein. Nucleic acid barcode molecule 1210 may comprise adapter sequence 1211, barcode sequence 1212 and adapter sequence 1213. Nucleic acid barcode molecule 1220 may comprise adapter sequence 1221, barcode sequence 1212, and adapter sequence 1223, wherein adapter sequence 1223 comprises a different sequence than adapter sequence 1213. In some instances, adapter 1211 and adapter 1221 comprise the same sequence. In some instances, adapter 1211 and adapter 1221 comprise different sequences. Although support 1230 is shown comprising nucleic acid barcode molecules 1210 and 1220, any suitable number of barcode molecules comprising common barcode sequence 1212 are contemplated herein. For example, in some embodiments, support 1230 further comprises nucleic acid barcode molecule 1250. Nucleic acid barcode molecule 1250 may comprise adapter sequence 1251, barcode sequence 1212 and adapter sequence 1253, wherein adapter sequence 1253 comprises a different sequence than adapter sequence 1213 and 1223. In some instances, nucleic acid barcode molecules (e.g., 1210, 1220, 1250) comprise one or more additional functional sequences, such as a UMI or other sequences described herein. The nucleic acid barcode molecules 1210, 1220 or 1250 may interact with analytes as described elsewhere herein, for example, as depicted in FIGS. 13A-C.

Referring to FIG. 13A, in an instance where cells are labelled with labeling agents, sequence 1323 may be complementary to an adapter sequence of a reporter oligonucleotide. Cells may be contacted with one or more reporter oligonucleotide 1310 conjugated labelling agents 1320 (e.g., polypeptide, antibody, or others described elsewhere herein). In some cases, the cells may be further processed prior to barcoding. For example, such processing steps may include one or more washing and/or cell sorting steps. In some instances, a cell that is bound to labelling agent 1320 which is conjugated to oligonucleotide 1310 and support 1330 (e.g., a bead, such as a gel bead) comprising nucleic acid barcode molecule 1390 is partitioned into a partition amongst a plurality of partitions (e.g., a droplet of a droplet emulsion or a well of a microwell array). In some instances, the partition comprises at most a single cell bound to labelling agent 1320. In some instances, reporter oligonucleotide 1310 conjugated to labelling agent 1320 (e.g., polypeptide, an antibody, pMHC molecule such as an MHC multimer, etc.) comprises a first adapter sequence 1311 (e.g., a primer sequence), a barcode sequence 1312 that identifies the labelling agent 1320 (e.g., the polypeptide, antibody, or peptide of a pMHC molecule or complex), and an adapter sequence 1313. Adapter sequence 1313 may be configured to hybridize to a complementary sequence, such as sequence 1323 present on a nucleic acid barcode molecule 1390. In some instances, oligonucleotide 1310 comprises one or more additional functional sequences, such as those described elsewhere herein.

Barcoded nucleic may be generated (e.g., via a nucleic acid reaction, such as nucleic acid extension or ligation) from the constructs described in FIGS. 13A-C. For example, sequence 1313 may then be hybridized to complementary sequence 1323 to generate (e.g., via a nucleic acid reaction, such as nucleic acid extension or ligation) a barcoded nucleic acid molecule comprising cell (e.g., partition specific) barcode sequence 1321 (or a reverse complement thereof) and reporter sequence 1312 (or a reverse complement thereof). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIG. 13A-C, or a combination of workflows for an individual analyte, as described elsewhere herein. For example, by using a combination of the workflows as generally depicted in FIGS. 13A-C, multiple analytes can be analyzed.

In some instances, analysis of an analyte (e.g. a nucleic acid, a polypeptide, a carbohydrate, a lipid, etc.) comprises a workflow as generally depicted in FIG. 13A. A nucleic acid barcode molecule 1390 may be co-partitioned with the one or more analytes. In some instances, nucleic acid barcode molecule 1390 is attached to a support 1330 (e.g., a bead, such as a gel bead), such as those described elsewhere herein. For example, nucleic acid barcode molecule 1390 may be attached to support 1330 via a releasable linkage 1340 (e.g., comprising a labile bond), such as those described elsewhere herein. Nucleic acid barcode molecule 1390 may comprise a barcode sequence 1321 and optionally comprise other additional sequences, for example, a UMI sequence 1322 (or other functional sequences described elsewhere herein). The nucleic acid barcode molecule 1390 may comprise a sequence 1323 that may be complementary to another nucleic acid sequence, such that it may hybridize to a particular sequence.

For example, sequence 1323 may comprise a poly-T sequence and may be used to hybridize to mRNA. Referring to FIG. 13C, in some embodiments, nucleic acid barcode molecule 1390 comprises sequence 1323 complementary to a sequence of RNA molecule 1360 from a cell. In some instances, sequence 1323 comprises a sequence specific for an RNA molecule. Sequence 1323 may comprise a known or targeted sequence or a random sequence. In some instances, a nucleic acid extension reaction may be performed, thereby generating a barcoded nucleic acid product comprising sequence 1323, the barcode sequence 1321, UMI sequence 1322, any other functional sequence, and a sequence corresponding to the RNA molecule 1360.

In another example, sequence 1323 may be complementary to an overhang sequence or an adapter sequence that has been appended to an analyte. For example, referring to FIG. 13B, in some embodiments, primer 1350 comprises a sequence complementary to a sequence of nucleic acid molecule 1360 (such as an RNA encoding for a BCR sequence) from an analyte carrier. In some instances, primer 1350 comprises one or more sequences 1351 that are not complementary to RNA molecule 1360. Sequence 1351 may be a functional sequence as described elsewhere herein, for example, an adapter sequence, a sequencing primer sequence, or a sequence the facilitates coupling to a flow cell of a sequencer. In some instances, primer 1350 comprises a poly-T sequence. In some instances, primer 1350 comprises a sequence complementary to a target sequence in an RNA molecule. In some instances, primer 1350 comprises a sequence complementary to a region of an immune molecule, such as the constant region of a TCR or BCR sequence. Primer 1350 is hybridized to nucleic acid molecule 1360 and complementary molecule 1370 is generated. For example, complementary molecule 1370 may be cDNA generated in a reverse transcription reaction. In some instances, an additional sequence may be appended to complementary molecule 1370. For example, the reverse transcriptase enzyme may be selected such that several non-templated bases 1380 (e.g., a poly-C sequence) are appended to the cDNA. In another example, a terminal transferase may also be used to append the additional sequence. Nucleic acid barcode molecule 1390 comprises a sequence 1324 complementary to the non-templated bases, and the reverse transcriptase performs a template switching reaction onto nucleic acid barcode molecule 1390 to generate a barcoded nucleic acid molecule comprising cell (e.g., partition specific) barcode sequence 1322 (or a reverse complement thereof) and a sequence of complementary molecule 1370 (or a portion thereof). In some instances, sequence 1323 comprises a sequence complementary to a region of an immune molecule, such as the constant region of a TCR or BCR sequence. Sequence 1323 is hybridized to nucleic acid molecule 1360 and a complementary molecule 1370 is generated. For example, complementary molecule 1370 may be generated in a reverse transcription reaction generating a barcoded nucleic acid molecule comprising cell (e.g., partition specific) barcode sequence 1322 (or a reverse complement thereof) and a sequence of complementary molecule 1370 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO20181075693, U.S. Patent Publication No. 2018/0105808, U.S. Patent Publication No. 2015/0376609, filed Jun. 26, 2015, and U S Patent Publication No 2019/0367969, each of which applications is herein entirely incorporated by reference for all purposes.

### EXAMPLES

Various features and embodiments of the disclosure are illustrated in the following representative examples, which are intended to be illustrative, and not limiting. Those skilled in the art will readily appreciate that the specific examples are only illustrative of the embodiments of the disclosure as described more fully in the claims which follow thereafter. Every embodiment and feature described in the application should be understood to be interchangeable and combinable with every embodiment contained within.

### Example 1: RNA Expression Assay of Fixed Jurkats Using Cold Protease Treatment in Combination with Catalytic Un-Fixing Agent of Compound (1)

This example illustrates the use of a low-temperature protease treatment in combination with the catalytic un-fixing agent of compound (1) to prepare a biological sample for bulk RNA expression assay from PFA-fixed Jurkats.

Materials and Methods: A protease stock solution of 100 mg/ml Subtilisin A from Bacillus licheniformis (Sigma-Aldrich, cat. #P5380) was prepared in H₂O and stored at−20° C. A stock solution of the un-fixing agent of 100 mM compound (1) (Cat. No. 419443; Sigma-Aldrich Corp., St. Louis, Mo., USA) in 30 mM Tris-HCl, 1 mM EDTA, pH 6.8, was prepared and stored at room temperature. Dissociated single cells (Jurkats) were pelleted by 400 g centrifugation for 5 minutes and the supernatant removed. A fixing reagent solution of 4% PFA in PBS with 0.2 U/μL Qiagen RNAse Inhibitor (Cat. No. 129916, QIAGEN) was added to the pelleted cells and the mixture incubated at 4° C. overnight. The resulting fixed cells were quenched with 10% FBS in PBS and spun down for 5 minutes at 500 g, 4° C. 150,000 fixed cells were washed once in PBS then resuspended in 100 μL, 30 mM Tris-HCL, 1 mM EDTA, pH 6.8. RNAse Inhibitor was added to the fixed cell solution together with one of the following: (a) 5 mg/mL Subtilisin A; or (b) 5 mg/mL Subtilisin A and 50 mM compound (1). The fixed cell solution with protease and with or without the un-fixing agent of compound (1) was allowed to incubate at 8° C. for 2 hours, followed by 15 minutes at 70° C. shaking continuously at 300 rpm on an Eppendorf Thermomixer. The resulting cell solutions were spun down for 5 minutes at 500 g, 4° C., and the supernatant and pellet fractions were collected separately. RNA extraction of the collected fractions was carried out using Qiagen 96 Kit (Cat. No. 74181, QIAGEN), bulk RNA sequencing, and/or single cell 3′ sequencing. Fresh cells, fresh cells with un-fixing conditions, and fixed cells without un-fixing treatment were also prepared and RNA extracted as controls. RNA yield was assessed by Qubit HS Assay (Q32855) and yield and DV200 quality metric was assessed by Agilent 4200 High Sensitivity ScreenTape (5067-5579).

Results: Results are summarized in Table 2. Relative to RNA recovery from fresh cells, the use of 2 h protease treatment at 8° C. allowed about 7% RNA recovery from 4% PFA-fixed Jurkats cells. The combination treatment with 50 mM compound (1) and protease for 2 h at 8° C., however, doubled the yield of RNA to about 15% of fresh, and improved the quality of recovered RNA as indicated by DV200. The addition of a 15 min, 70° C. degree step after the 2 h, 8° C. treatment to the combination treatment with 50 mM compound (1) and protease led to a substantially improved 71% recovery of RNA (relative to fresh) with >95% DV200.

TABLE 2

| | Pellet | | | | | Supernatant | | | | |
|---|---|---|---|---|---|---|---|---|---|---|
| | Qubit | | Tapestation | | | Qubit | | Tapestation | | |
| | Avg | | Avg | | | Avg | | Avg | | |
| | (SD) | | (SD) | | DV200 | (SD) | | (SD) | | DV200 |
| Fresh | 413 | (8) | 413 | (8) | 87.2 | | | | | |
| Protease | 21 | (1) | 21 | (2) | 61.0 | | | 8 | (3) | 52.5 |
| 8° C./2 hr | | | | | | | | | | |
| Protease + | | | 12 | (2) | 62.9 | 43 | (4) | 54 | (7) | 86.5 |
| Compound (1) | | | | | | | | | | |
| 8° C./2 hr | | | | | | | | | | |
| Protease + | 65 | (9) | 49 | (15) | 66.3 | 230 | (17) | 246 | (22) | 95.2 |
| Compound (1) | | | | | | | | | | |
| 8° C./2 hr | | | | | | | | | | |
| 70° C./15 min | | | | | | | | | | |

### Example 2: Bulk RNA Sample Preparation from Fixed Cells Using a Cold Protease Treatment and Catalytic Un-Fixing Agents of Compounds (1) and (8)

This example illustrates a study of the use of the catalytic un-fixing agent of compound (8) in alone or in combination with the un-fixing agent of compound (1) and a low-temperature protease treatment to un-fix PFA-fixed cells (Jurkats) and measure release of RNA from the cells into the pellet and/or supernatant.

Materials and Methods:

A. Protease preparation: A protease stock solution of 100 mg/ml Subtilisin A from Bacillus licheniformis (Sigma-Aldrich, cat. #P5380) was prepared in H₂O and stored at−20° C.

B. Unfixing agent of compound (8): The un-fixing agent of compound (8) was prepared using the following 2-step synthesis procedure.

Step 1: Diethyl (4-aminopyridin-3-yl)phosphonate. In step 1 the compound, diethyl (4-aminopyridin-3-yl)phosphonate was prepared according to the procedure described in Guilard, R. et al. Synthesis, 2008, 10, 1575-1579. Briefly, to a solution of 3-bromopyridine-4-amine (2.5 g, 14.5 mmol, 1 equiv) (CAS: 13534-98-0, Sigma Aldrich) in ethanol (58 mL) was added diethyl phosphite (2.2 mL, 17.3 mmol, 1.2 equiv.) triethylamine (3 mL, 1.5 equiv), PPh₃ (1.1 g, 4.3 mmol, 30 mol %) and Pd(OAc)₂ (0.39 g, 1.73 mmol, 12 mol %). The reaction mixture was purged with Argon for 5 min. After heating to reflux for 24 h, the reaction mixture was cooled to RT and conc. in vacuo. The residue was purified by silica gel chromatography (MeOH/DCM) to give the title compound (0.35 g, 11% yield). ¹HNMR (80 MHz, CDCl₃): □=1.15 (t, 6H, CH₃), 4.18-3.69 (m, 4H, CH₂), 5.99 (br-s, 2H, NH₂), 6.49 (d, 1H), 8.03-7.93 (m, 1H), 8.22 (d, 1H).

Step 2: 4-Aminopyridin-3-yl)phosphonic acid (compound (8). In step 2, the target compound, (4-Aminopyridin-3-yl)phosphonic acid (compound (8)) was prepared by acid hydrolysis of the precursor compound of step 1. Diethyl (4-aminopyridin-3-yl)phosphonate (0.35 g, 1.52 mmol, 1 equiv) was suspended in 6 N HCl (aq.) (8 mL). After refluxing for 12h, the reaction mixture was concentrated in vacuo. The residue was washed with DCM, ether and conc in vacuo to afford the target compound (8) (247 mg, 93% yield). ¹HNMR (80 MHz, D₂O): □=6.85-6.55 (m, 1H), 8.05-7.94 (m, 1H), 8.40-8.26 (m, 1H).

C. Un-fixing agent stock solutions: A stock solution of the un-fixing agent of 100 mM compound (1) (Cat. No. 419443; Sigma-Aldrich Corp., St. Louis, Mo., USA) in 30 mM Tris-HCl, 1 mM EDTA, pH 6.8, was prepared and stored at room temperature. A stock solution of the un-fixing agent of 100 mM compound (8) (prepared as described above) in 30 mM Tris-HCl, 1 mM EDTA, pH 6.8, was prepared and stored at room temperature.

D. Fixed cell preparation: Dissociated single cells (Jurkats) were pelleted by 400 g centrifugation for 5 minutes and the supernatant removed. A fixing reagent solution of 4% PFA in PBS with 0.2 U/μL Qiagen RNAse Inhibitor (Qiagen, cat. #129916) was added to the pelleted cells and the mixture incubated at 4° C. overnight. The resulting fixed cells were quenched with 10% FBS in PBS and spun down for 5 minutes at 500 g, 4° C. 150,000 fixed cells were washed once in PBS then resuspended in 100 μL, 30 mM Tris-HCL, 1 mM EDTA, pH 6.8.

E. Cell un-fixing/protease treatment: Inhibitor was added to the fixed cell solution together with one of the following: (a) 5 mg/mL Subtilisin A protease; (b) 5 mg/mL Subtilisin A protease and 50 mM compound (1); (c) 5 mg/mL Subtilisin A protease and 50 mM compound (8); (d) 5 mg/mL Subtilisin A protease and 25 mM compound (8) and 25 mM compound (1). The fixed cell solutions with protease and with or without the un-fixing agents of compounds (1) and/or (8) were allowed to incubate at 8° C. for 2 hours, followed by 15 minutes at 70° C. shaking continuously at 300 rpm on an Eppendorf Thermomixer. The resulting cell solutions were spun down for 5 minutes at 500 g, 4° C., and the supernatant and pellet fractions were collected separately.

F. RNA quantitation: RNA extraction of the collected fractions was carried out using Qiagen 96 Kit (Qiagen, cat. #74181), bulk RNA sequencing, and/or single cell 3′ sequencing. Fresh cells, fresh cells with un-fixing conditions, and fixed cells without un-fixing treatment were also prepared and RNA extracted as controls. RNA yield was assessed by Qubit HS Assay (Q32855) and yield and DV200 quality metric was assessed by Agilent 4200 High Sensitivity ScreenTape (5067-5579).

Results: Results are summarized in Table 3. Relative to RNA recovery from fresh cells, the use of 50 mM compound (8) in combination with Subtilisin A protease for 2h at 8° C. followed by 15 min at 70° C. yielded 100% of RNA (relative to fresh) in the pellet. The quality of the RNA recovered in the pellet as indicated by DV200.

TABLE 3

| | Pellet | | | | | Supernatant | | | | |
|---|---|---|---|---|---|---|---|---|---|---|
| | Qubit | | Tapestation | | | Qubit | | Tapestation | | |
| | Avg | | Avg | | | Avg | | Avg | | |
| | (SD) | | (SD) | | DV200 | (SD) | | (SD) | | DV200 |
| Fresh | 1350 | (150) | 1805 | (460) | 90.7 | | | 0 | | |
| Protease | 108.4 | (0.6) | 49 | (3) | 74.1 | | | 0 | | |
| 8° C./2 hr | | | | | | | | | | |
| Protease | 106 | (90) | 58 | (43) | 47.3 | 398 | (260) | 231 | (204) | 60.5 |
| 8° C./2 hr | | | | | | | | | | |
| 70° C./15 min | | | | | | | | | | |
| Protease + | 167 | (10) | 96 | (33) | 44.5 | 238 | (76) | 126 | (16) | 75.3 |
| Compound (1) | | | | | | | | | | |
| 8° C./2 hr | | | | | | | | | | |
| 70° C./15 min | | | | | | | | | | |
| Protease + | 1566 | (0) | 745 | (60) | 83.4 | | | | | |
| Compound (8) | | | | | | | | | | |
| 8° C./2 hr | | | | | | | | | | |
| 70° C./15 min | | | | | | | | | | |
| Protease + | 595 | (270) | 261 | (61) | 81.1 | 492 | (17) | 290 | (20) | 92.4 |
| Compound (1) + | | | | | | | | | | |
| Compound (8) | | | | | | | | | | |
| 8° C./2 hr | | | | | | | | | | |
| 70° C./15 min | | | | | | | | | | |
| Protease + | 545 | (56) | 305 | (36) | 65.4 | 410 | (53) | 224 | (25) | 87.0 |
| Compound (1) + | | | | | | | | | | |
| Compound (8) | | | | | | | | | | |
| 53° C./45 min | | | | | | | | | | |
| Protease + | 383 | (135) | 182 | (18) | 68.6 | 669 | (134) | 266 | (48) | 79.4 |
| Compound (1) + | | | | | | | | | | |
| Compound (8) | | | | | | | | | | |
| 53° C./45 min | | | | | | | | | | |
| 70° C./15 min | | | | | | | | | | |

### Example 3: Bulk Sequencing of RNA from Fixed Cells Using a Cold Protease Treatment and Catalytic Un-Fixing Agents of Compounds (1) and (8)

This example illustrates a study of bulk sequencing of RNA from PFA-fixed cells treated Subtilisin A at low temperature or Proteinase K at 53 C, and the un-fixing agents of compounds (1) and/or (8), relative to bulk sequencing of RNA from fresh cells.

Materials and Methods:

A. Protease preparation: A Subtilisin A protease stock solution was prepared as in Example 2. A stock solution of 20 mg/mL Proteinase K (Sigma-Aldrich, cat. #AM258) was prepared in H₂O and stored at −20° C.

B. Un-fixing agent stock solutions: Stock solutions of the un-fixing agents of compound (1) and compound (8) were prepared as in Example 2.

C. Fixed cell preparation: Dissociated single cells (Jurkats) were pelleted by 400 g centrifugation for 5 minutes and the supernatant removed. A fixing reagent solution of 4% PFA in PBS with 0.2 U/μL Qiagen RNAse Inhibitor (Qiagen, cat. #129916) was added to the pelleted cells and the mixture incubated at 4° C. overnight. The resulting fixed cells were quenched with 10% FBS in PBS and spun down for 5 minutes at 500 g, 4° C. 150,000 fixed cells were washed once in PBS then resuspended in 100 μL, 30 mM Tris-HCL, 1 mM EDTA, pH 6.8.

D. Cell un-fixing/protease treatment: RNAse Inhibitor was added to the fixed cell solution together with (a) 5 mg/mL Subtilisin A protease and 25 mM compound (8) and 25 mM compound (1); or (b) 0.1 mg/mL Proteinase K protease and 25 mM compound (1) and 25 mM compound (8). The fixed cell solutions with Subtilisin A and the un-fixing agents of compounds (1) and/or (8) were allowed to incubate at 8° C. for 2 hours, followed by 15 minutes at 70° C. shaking continuously at 300 rpm on an Eppendorf Thermomixer. The fixed cell solutions treated with Proteinase K protease and with the un-fixing agents of compounds (1) and/or (8) were allowed to incubate 53° C. for 45 min followed by 15 minutes at 70° C. shaking continuously at 300 rpm on an Eppendorf Thermomixer. The resulting cell solutions were spun down for 5 minutes at 500 g, 4° C., and the supernatant and pellet fractions were collected separately.

E. RNA Isolation: RNA extraction of the collected fractions was carried out using Qiagen 96 Kit (Qiagen, cat. #74181). Control samples of fresh cells, fresh cells with un-fixing conditions, and fixed cells without un-fixing treatment were also prepared and RNA extracted. RNA yield was assessed by Qubit HS Assay (Q32855) and yield and DV200 quality metric was assessed by Agilent 4200 High Sensitivity ScreenTape (5067-5579).

F. Bulk RNA sequencing: cDNA amplification of un-fixing agent treated and control samples was performed using an equivalent of 10 ng RNA. Bulk RNA was loaded in master mix in substitute for a single cell suspension, then GEM-RT, post cDNA amplification, and library prep was performed according to the 10× Genomics Single Cell 3′V3 protocol (10× Genomics, Pleasanton, Calif., USA). A 3000-cell load of fresh cells was used as a single cell reference (Fresh SC3P) and library prep was performed using Single cell 3′V3 protocol (10× Genomics, Pleasanton, Calif., USA). The final libraries were sequenced to between 25 and 100 million reads on a NovaSeq 6000 sequencer (Illumina Inc., San Diego, Calif., USA). Bulk library complexity was estimated using the software package Preseq, as described by Daley and Smith (see e.g., Daley and Smith, “Predicting the molecular complexity of sequencing libraries,” Nature Methods 10:325-327, 2013). Library complexity as used here refers to the estimated number of unique RNA molecules aligned properly to the transcriptome (i.e., reads considered to be informative and used for gene expression counting) as a function of all sequenced reads. Gene expression counts were down-sampled across libraries to match the lowest sequencing depth and pairwise gene expression correlations were computed as the Pearson correlation (R²) of gene expression counts between samples. When comparing gene expression data from control, unfixed cells, gene expression counts were summed across cells to produce pseudo-bulk gene expression counts, as is customary in commercial gene expression analysis software (e.g., 10× Genomics, Pleasanton, Calif., USA).

Results: As shown by the RNA quantitation and quality results summarized in Table 4 below, the fixed sample treated using Subtilisin A at low temperature together with the un-fixing agents of compound (1) and (8) resulted in a higher cDNA yield together with higher relative fraction of UMIs and high R2 for gene expression relative to a fresh sample.

TABLE 4

| | | Relative fraction | R² for Gene |
|---|---|---|---|
| | cDNA | of Max. No. | Expression |
| | yield | UMIs to Fresh | Fresh v. |
| | (SD) | (SD) | Un-fixed |
| Fresh | 182 | 1 | 1 |
| | (20) | (0.02) | |
| Compound (1) + | 37 | 0.56 | .70 |
| Compound (8) + | (20) | (0.04) | |
| 0.1 mg/mL Proteinase K | | | |
| 53° C./45 min | | | |
| Compound (1) + | 55 | 0.68 | .72 |
| Compound (8) + | (29) | (0.03) | |
| Subtilisin A | | | |
| 8° C./2 h + 70° C./15 min | | | |

### Example 4: Bulk Un-Fixing of PFA-Fixed PBMCs with Compound (8) and a Cold-Active Protease Followed by Single-Cell Partition Barcoding and cDNA Synthesis

This example illustrates a study of bulk low-temperature un-fixing of PFA-fixed cells using the un-fixing agent of compound (8) and a cold-active protease (e.g., ArcticZymes Proteinase) at 14 C or 25 C, followed by protease deactivation, partitioning of un-fixed cells into GEMs with barcoding, and reverse transcription of the un-fixed RNA to provide cDNA.

Materials and Methods:

A. Protease preparation: A stock solution of 10 U/mL of the cold-active protease, ArcticZymes Proteinase (ArcticZymes Technologies ASA, Tromso, Norway) was stored at−20° C.

B. Un-fixing agent of compound (8) stock solutions: A stock solution of the un-fixing agent of 300 mM compound (8) in 50 mM Tris-HCl, 1 mM EDTA, pH 8.3, was prepared, filtered using a 5 μm syringe filter, and stored at room temperature.

C. Fixed cell preparation: Isolated single cells (PBMCs) were pelleted by 400 g centrifugation for 5 minutes and the supernatant removed. A fixing reagent solution of 4% PFA in PBS with 0.2 U/μL Qiagen RNAse Inhibitor (Qiagen, cat. #129916) was added to the pelleted cells and the mixture incubated at 4° C. overnight. The resulting fixed cells were quenched with RNAse-free 10% FBS (Seradigm 97069-085) in PBS and spun down for 5 minutes at 500 g, 4° C. 150,000 fixed cells were washed once in PBS then resuspended in 0.4% RNase free BSA in PBS with 20 U/mL RNase inhibitor.

D. Cell un-fixing/protease treatment: RNAse Inhibitor was added to the fixed cell solution together with 10 U/mL of the cold-active protease, ArcticZymes Proteinase, 50-200 mM of the un-fixing agent, compound (8), and 1 mM of the protease inhibitor, PMSF. The fixed cell solution treated with the protease and compound (8) was allowed to incubate at 14-25° C. for 45-90 min, followed by an incubation at 70-85° C. for 15 min. The resulting cell solution was spun down for 5 minutes at 500 g, 4° C., and the supernatant and pellet fractions were collected separately. Microscopic imaging showed that the cells un-fixed by this treatment remained intact although somewhat swollen relative to the fresh or PFA-fixed cells.

E. Partitioning of pellet fractions into GEMs and 3′-RT: pellet fractions collected from the un-fixing/protease treatment were centrifuged at 5 min 300 g and washed with PBS 0.04% BSA twice before loaded into the Single Cell 3′V3 protocol standard master mix used with the Chromium System (10× Genomics, Pleasanton, Calif., USA) for partitioning samples together with barcoded gel beads in discrete droplets called GEMs (“Gel Beads in Emulsion”). Once generated, the GEMs are collected, and a heat incubation step is carried out. The heating step facilitates release of the cell contents and RNA, capture of RNA by barcode oligonucleotides, and the reverse-transcription (RT) reaction that results in cDNA synthesis incorporating the barcodes in the 3′ synthons.

cDNA electropherogram analysis was performed using Agilent 2100 Bioanalyzer 5067-4626 to assess DNA size and yield from each sample.

Determination and mapping of PBMC cell types present in the samples was carried out as follows: PBMC cell type determination was performed by automated meta-analysis of cell clusters identified using differentially expressed marker gene expression. PBMC cell type composition was identified by an automated script that quantifies the number and fraction of cell types known to be detected in PBMC samples by categorizing cells based on a combination of differentially expressed known marker genes for each cell type, with unclassified cells going to the undetermined category.

Results:

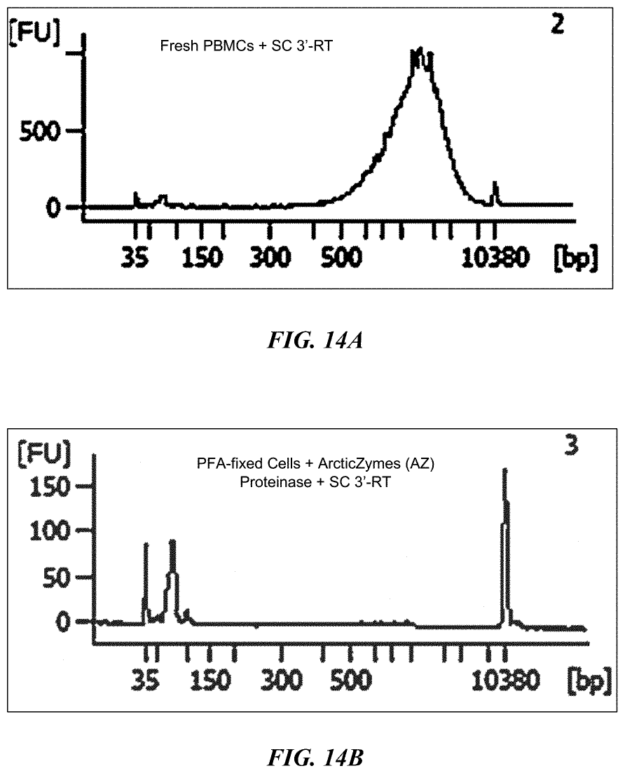

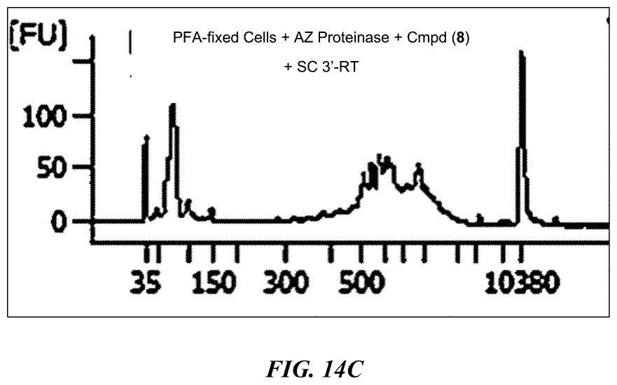

As shown by the plots depicted in FIGS. 14A, 14B and 14C, the cDNA synthons generated using a treatment with the cold-active ArcticZymes Proteinase incubated at 14-25 C for 45-90 min and the un-fixing agent of compound (8), exhibited a cDNA electropherogram profile (FIG. 14C) that more closely resembling the profile for Fresh cells (FIG. 14A), than the profile from the treatment without compound (8) (FIG. 14B), which indicates little or no cDNA obtained from the sample. Additionally, the correlation of the gene expression values (R²) for Fresh and Fixed+un-fixing treatment was substantially higher than for Fresh and Fixed without any un-fixing treatment.

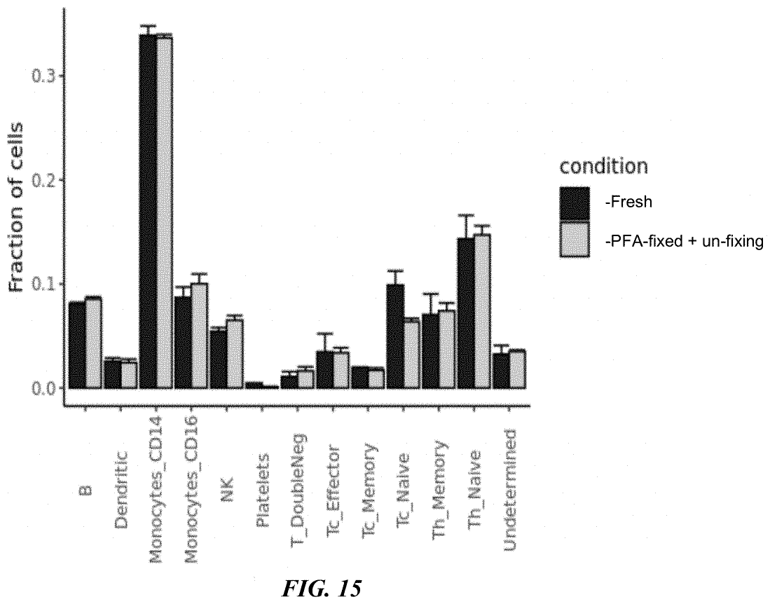

Additionally, as shown by the plot depicted in FIG. 15, cell counting was carried out to determine the proportion of different PBMC cell types found in Fresh cells as compared to Fixed cells subjected to the cold-active protease and un-fixing agent treatment. It was observed that the proportions of B cells, monocytes, T cells, and dendritic cells found in the Fresh cell sample was similar to the proportions found in the Fixed cell samples subjected to the cold-active protease and un-fixing agent treatment. These comparative PBMC cell counting results indicate that the treatment using a cold-active protease and an unfixing agent can result in recovery relatively rare cell types from fixed samples and that allows analysis these cell types in a droplet-based assay.

While the foregoing disclosure has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the disclosure are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A method for preparing a biological sample comprising incubating a solution of a fixed biological sample and a protease at a temperature of between about 5° C. and about 15° C. for at least an hour, wherein the solution optionally further comprises an un-fixing agent.

2. The method of claim 1, wherein the fixed biological sample comprises a plurality of fixed single cells.

3. The method of any one of claims 1-2, wherein the fixed biological sample has been fixed with paraformaldehyde (“PFA”); optionally, fixed with PFA at a concentration of 1%−4%.

4. The method of any one of claims 1-3, wherein the protease and the un-fixing agent are capable of removing crosslinks formed in biomolecules by fixation with PFA.

5. The method of any one of claims 1-4, wherein the un-fixing agent is a composition comprising a compound selected from compound (1), compound (2), compound (3), compound (4), compound (5), compound (6), compound (7), compound (8), compound (9), compound (10), compound (11), compound (12), compound (13), compound (14), compound (15), or a combination thereof; optionally, wherein the un-fixing agent is a composition comprising a compound selected from compound (1), compound (8), or a combination thereof.

6. The method of any one of claims 1-5, wherein the protease is a cold-active protease.

7. The method of any one of claims 1-6, wherein the protease has an average activity of at least 1.0 Units/mg of protease at a temperature of between about 5° C. and about 15° C.

8. The method of any one of claims 1-7, wherein the protease has maximum activity at a temperature of between about 50° C. and about 60° C.

9. The method of any one of claims 1-8, wherein the protease concentration in the solution is between about 1 mg/mL and 100 mg/mL; optionally, the protease concentration in the solution is between about 5 mg/mL and 10 mg/mL.

10. The method of any one of claims 1-9, wherein subsequent to incubating the solution is shaken at a temperature of between about 65° C. and 75° C. for at least 15 minutes.

11. The method of any one of claims 1-10, wherein the protease is a serine protease (E.C. 3.4.21); optionally, wherein the serine protease is selected from chymotrypsin-like, trypsin-like, thrombin-like, elastase-like, and subtilisin-like.

12. The method of any one of claims 1-11, wherein the protease is selected from: alcalase, alkaline proteinase, ArcticZymes Proteinase, bacillopeptidase A, bacillopeptidase B, bioprase, colistinase, esperase, genenase, kazusase, maxatase, proteinase K, protease S, savinase, Serratia peptidase, subtilisin A, subtilisin B, subtilisin BL, subtilisin E, subtilisin J, subtilisin S, subtilisin S41, thermoase, trypsin, and a combination thereof.

13. The method of any one of claims 1-12, wherein the protease is a non-naturally occurring protease.

14. The method of any one of claims 1-13, wherein the fixed biological sample is derived from a tissue sample, a biopsy sample, or a blood sample.

15. The method of any one of claims 1-14, wherein the fixed biological sample comprises one or more single cells.

16. The method of any one of claims 1-15, wherein the amount of time prior to incubating the solution when the biological sample is fixed is at least 1 hour, at least 2 hours, at least 6 hours, at least 12 hours, at least 24 hours, at least 1 week, at least 1 month, at least 6 months, or longer.

17. The method of any one of claims 1-16, wherein the method further comprises generating a discrete droplet encapsulating the biological sample.

18. The method of any one of claims 1-16, wherein the method further comprises generating a discrete droplet encapsulating the fixed biological sample and the protease.

19. The method of any one of claims 2-16, wherein the method further comprises generating a discrete droplet encapsulating the fixed biological sample, the protease, and the un-fixing agent.

20. The method of any one of claims 17-19, wherein the discrete droplet further comprises assay reagents; optionally, wherein the assay reagents are contained in a bead.

21. The method of any one of claims 17-20, wherein the discrete droplet further comprises a barcode; optionally, wherein the barcode contained in a bead.

22. An assay method comprising:
(a) preparing a biological sample by incubating a solution of a fixed biological sample and a protease at a temperature of between about 5° C. and about 15° C. for at least an hour, wherein the solution optionally further comprises an un-fixing agent;
(b) contacting the biological sample with assay reagents; and
(c) detecting analytes from the reaction of the assay reagents and the biological sample.

23. The assay method of claim 22, wherein the method further comprises generating a discrete droplet encapsulating the biological sample and assay reagents.

24. The assay method of any one of claims 22-23, wherein the fixed biological sample has been fixed with paraformaldehyde (“PFA”); optionally, fixed with PFA at a concentration of 1%-4%.

25. The assay method of any one of claims 22-24, wherein the protease and the un-fixing agent are capable of removing crosslinks formed in biomolecules by fixation with PFA.

26. The assay method of any one of claims 22-25, wherein the un-fixing agent is a composition comprising a compound selected from compound (1), compound (2), compound (3), compound (4), compound (5), compound (6), compound (7), compound (8), compound (9), compound (10), compound (11), compound (12), compound (13), compound (14), compound (15), or a combination thereof; optionally, wherein the un-fixing agent is a composition comprising a compound selected from compound (1), compound (8), or a combination thereof.

27. The assay method of any one of claims 22-25, wherein the protease is a cold-active protease.

28. The assay method of any one of claims 22-27, wherein the protease has an average activity of at least 1.0 Units/mg of protease at a temperature of between about 5° C. and about 15° C.

29. The assay method of any one of claims 22-28, wherein the protease has maximum activity at a temperature of between about 50° C. and about 60° C.

30. The assay method of any one of claims 22-29, wherein the protease concentration in the solution is between about 1 mg/mL and 100 mg/mL; optionally, the protease concentration in the solution is between about 5 mg/mL and 10 mg/mL.

31. The assay method of any one of claims 22-30, wherein subsequent to incubating the solution is shaken at a temperature of between about 65° C. and 75° C. for at least 15 minutes.

32. The assay method of any one of claims 22-31, wherein the protease is a serine protease (E.C. 3.4.21); optionally, wherein the serine protease is selected from chymotrypsin-like, trypsin-like, thrombin-like, elastase-like, and subtilisin-like.

33. The assay method of any one of claims 22-32, wherein the protease is selected from: alcalase, alkaline proteinase, ArcticZymes Proteinase, bacillopeptidase A, bacillopeptidase B, bioprase, colistinase, esperase, genenase, kazusase, maxatase, proteinase K, protease S, savinase, Serratia peptidase, subtilisin A, subtilisin B, subtilisin BL, subtilisin E, subtilisin J, subtilisin S, subtilisin S41, thermoase, trypsin, and a combination thereof.

34. The assay method of any one of claims 22-33, wherein the protease is a non-naturally occurring protease.

35. The assay method of any one of claims 22-34, wherein the fixed biological sample is derived from a tissue sample, a biopsy sample, or a blood sample.

36. The assay method of any one of claims 22-35, wherein the fixed biological sample comprises one or more single cells.

37. An assay method comprising:
(a) incubating a solution comprising a fixed biological sample and a protease at a temperature of between about 5° C. and about 15° C. for at least an hour, wherein the solution optionally further comprises an un-fixing agent;
(b) heating the solution of step (a) to 70 C for 15 minutes;
(c) centrifuging the solution of step (b) to obtain a pellet comprising cells of an un-fixed biological sample;
(d) resuspending the cells from the pellet in a solution;
(e) generating a discrete droplet encapsulating a cell from the pellet of step (d) and assay reagents; and
(e) detecting analytes from the reaction of the cell from the pellet and the assay reagent.

38. The assay method of claim 37, wherein the fixed biological sample has been fixed with paraformaldehyde (“PFA”); optionally, fixed with PFA at a concentration of 1%-4%.

39. The assay method of any one of claims 37-38, wherein the un-fixing agent is a composition comprising a compound selected from compound (1), compound (2), compound (3), compound (4), compound (5), compound (6), compound (7), compound (8), compound (9), compound (10), compound (11), compound (12), compound (13), compound (14), compound (15), or a combination thereof.

40. The assay method of claim 39, wherein the un-fixing agent composition comprises compound (1), compound (8), or a combination thereof.

41. The assay method of any one of claims 37-40, wherein the protease is a cold-active protease.

42. The assay method of any one of claims 37-41, wherein the protease has an average activity of at least 1.0 Units/mg of protease at a temperature of between about 5° C. and about 15° C.

43. The assay method of any one of claims 37-42, wherein the protease has maximum activity at a temperature of between about 50° C. and about 60° C.

44. The assay method of any one of claims 37-43, wherein the protease concentration in the solution is between about 1 mg/mL and 100 mg/mL; optionally, the protease concentration in the solution is between about 5 mg/mL and 10 mg/mL.

45. The assay method of any one of claims 37-44, wherein the protease is a serine protease (E.C. 3.4.21); optionally, wherein the serine protease is selected from chymotrypsin-like, trypsin-like, thrombin-like, elastase-like, and subtilisin-like.

46. The assay method of any one of claims 37-45, wherein the protease is selected from: alcalase, alkaline proteinase, ArcticZymes Proteinase, bacillopeptidase A, bacillopeptidase B, bioprase, colistinase, esperase, genenase, kazusase, maxatase, proteinase K, protease S, savinase, Serratia peptidase (i.e., peptidase derived from Serratia sp.), subtilisin A, subtilisin B, subtilisin BL, subtilisin E, subtilisin J, subtilisin S, subtilisin S41, thermoase, trypsin, and a combination thereof.

47. The assay method of any one of claims 37-46, wherein the protease is a non-naturally occurring protease.

48. The assay method of any one of claims 37-47, wherein the fixed biological sample is derived from a tissue sample, a biopsy sample, or a blood sample.

49. A kit comprising:
assay reagents;
an un-fixing agent composition; and
a protease composition.

50. The kit of claim 49, wherein the protease is a cold-active protease.

51. The kit of any one of claims 49-50, wherein the protease has an average activity of at least 1.0 Units/mg of protease at a temperature of between about 5° C. and about 15° C.

52. The kit of any one of claims 49-51, wherein the protease is selected from: alcalase, alkaline proteinase, ArcticZymes Proteinase, bacillopeptidase A, bacillopeptidase B, bioprase, colistinase, esperase, genenase, kazusase, maxatase, proteinase K, protease S, savinase, Serratia peptidase, subtilisin A, subtilisin B, subtilisin BL, subtilisin E, subtilisin J, subtilisin S, subtilisin S41, thermoase, trypsin, and a combination thereof.

53. The kit of any one of claims 49-52, wherein the unfixing agent composition comprises a compound selected from compound (1), compound (2), compound (3), compound (4), compound (5), compound (6), compound (7), compound (8), compound (9), compound (10), compound (11), compound (12), compound (13), compound (14), compound (15), or a combination thereof.

54. The kit of any one of claims 49-53, wherein the un-fixing agent composition is contained in a bead.

55. The kit of any one of claims 49-54, wherein the assay reagents are contained in a bead.

56. The kit of any one of claims 49-55, wherein the assay reagents comprise a barcode.

57. The kit of any one of claims 49-56, wherein the kit further comprises a fixing reagent;
optionally, wherein the fixing reagent is a solution of 1%-4% PFA.
